(19) United States
(12) Patent Application Publication
(10) Pub. No.: US 2025/0163026 A1
(43) Pub. Date: May 22, 2025

(54) Deuterium-Enriched Nirmatrelvir as SARS-CoV-2 Mpro Inhibitor for the Treatment of COVID-19

(71) Applicant: Daljit Singh Dhanoa, Del Mar, CA (US)

(72) Inventor: Daljit Singh Dhanoa, Del Mar, CA (US)

(21) Appl. No.: 18/516,928

(22) Filed: Nov. 21, 2023

(51) Int. Cl. C07D 403/12; A61K 31/706; A61K 45/06; A61P 31/14; C07B 59/00

(52) U.S. Cl. CPC C07D 403/12; A61K 31/706; A61K 45/06; A61P 31/14; C07B 59/002; C07B 2200/05

(57) ABSTRACT

The present invention is concerned with novel deuterium-enriched compounds of the general chemical structural formula I., and pharmaceutically acceptable salts, compositions, and methods of use thereof,

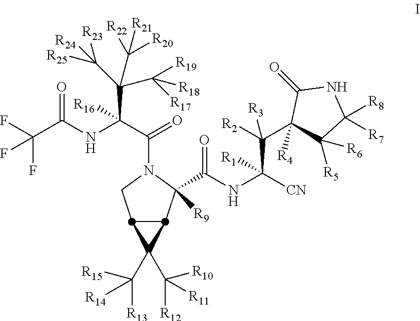

wherein, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅ are independently D (deuterium), H (hydrogen). The compounds of general chemical formula I are novel deuterium-enriched analogs of the SARS-CoV-2 main protease (M^{Pro}) inhibitor Nirmatrelvir for the treatment of COVID-19 and related diseases caused by various coronaviruses and their variants.

### SUMMARY OF THE INVENTION

The present invention is concerned with novel deuterium-enriched compounds of the general structural formula I., and pharmaceutically acceptable salts, compositions, and methods of use

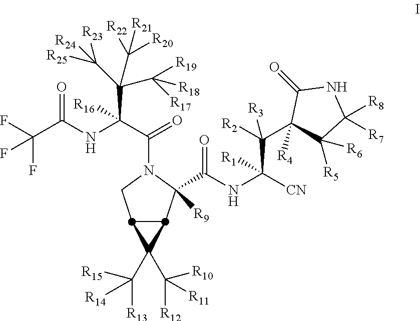

there of, wherein,
R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅ are independently D (deuterium), H (hydrogen).

The compounds of Formula I are novel deuterium containing analogs of the SARS-CoV-2 (COVID-19) antiviral drug Nirmatrelvir (I, wherein R₁-R₂₅ are H, hydrogen atom).

The compounds of this invention represented by chemical structural formula I [(wherein R₁-R₂₅ are independently deuterium (D) or hydrogen (H)] and are useful for the prevention and treatment of COVID-19 disease caused by the coronavirus Severe Acute Respiratory Syndrome Coronavirus-2 (SARS-CoV-2), and Severe Acute Respiratory Syndrome Coronavirus (SARS-CoV), Middle East respiratory syndrome coronavirus (MERS-CoV), and their variants.

These compounds are potent and selective Severe Acute Respiratory Syndrome Coronavirus-2 (SARS-CoV-2) main protease (M^{Pro}) inhibitors and prevent viral replication and are useful in the treatment of the Covid-19 disease, Severe Acute Respiratory Syndrome-Coronavirus (SARS-CoV), the Middle East respiratory syndrome coronavirus (MERS-CoV), and other diseases caused by various coronaviruses, and their variants.

The compounds of Formula I, their enantiomers, diastereomers, and pharmaceutical salts and combinations thereof, have inhibitory activity for Severe Acute Respiratory Syndrome Coronavirus-2 (SARS-CoV-2), Severe Acute Respiratory Syndrome-Coronavirus (SARS-CoV), Middle East Respiratory Syndrome (MERS-CoV), and are particularly useful for the prevention and treatment of the life-threatening disease Covid-19, Severe Acute Respiratory Syndrome Coronavirus-2 (SARS-CoV-2, Severe Acute Respiratory Syndrome-Coronavirus (SARS-CoV), Middle East Respiratory Syndrome (MERS-CoV), and their variants.

This invention further constitute a method for inhibiting the coronavirus enzyme, Severe Acute Respiratory Syndrome Coronavirus-2 (SARS-CoV-2) main protease (M^{Pro}, also referred to as 3CL protease), Severe Acute Respiratory Syndrome-Coronavirus (SARS-CoV), Middle East Respiratory Syndrome (MERS-CoV), in mammal, including humans, which comprises administering to a mammal in need of such treatment an effective amount of a compound of structural Formula I.

These novel (SARS-CoV-2) inhibitors (or SARS-CoV-2 M^{Pro}) are designed by applying our robust drug design and discovery chemistry, innovative structure-based drug design, medicinal chemistry, and deuterium chemistry.

The present invention provides methods of inhibiting the activity of the SARS (CoV-2) M^{Pro} protease, comprising contacting the coronavirus SARS (CoV-2) or COVID-19 disease, with an effective amount of a SARS (CoV-2) M^{Pro} protease inhibitor compound or agent.

In one embodiment of the present invention, the SARS coronavirus M^{Pro} protease inhibitor is administered orally or intravenously.

The present invention also provides a method of treating a condition that is mediated by coronavirus SARS (CoV-2) activity in a patient by administering to said patient a pharmaceutically effective amount of a SARS protease inhibitor.

The present invention also provides a method of targeting SARS inhibition as a means of treating indications caused by SARS (COVID-19)-related viral infections.

The present invention also provides a method of identifying cellular or viral pathways interfering with the functioning of the members of which could be used for treating indications caused by SARS infections by administering a SARS protease inhibitor.

The present invention also provides a method of using SARS protease inhibitors as tools for understanding mechanism of action of other SARS inhibitors.

The present invention also provides a method of using SARS(CoV-2) M^{Pro} or SARS(CoV-2) 3CL protease inhibitors for carrying out gene profiling experiments for monitoring the up or down regulation of genes for the purpose of identifying inhibitors for treating indications caused by SARS (COVID-19) or SARS like infections.

The present invention further provides a pharmaceutical composition for the treatment of SARS (COVID-19) in a mammal containing an amount of a SARS(CoV-2) M^{Pro} or SARS(CoV-2) 3CL protease inhibitor that is effective in treating SARS and a pharmaceutically acceptable carrier.

### BACKGROUND OF THE INVENTION

The worldwide outbreak of COVID-19 caused by a novel coronavirus, severe acute respiratory syndrome coronavirus 2 (SARS-CoV-2) has become a global pandemic since early 2020. The two previous outbreaks with the highly pathogenic Severe Acute Respiratory Syndrome-Coronavirus (SARS-CoV), the Middle East respiratory syndrome coronavirus (MERS-CoV), were quickly suppressed through rigorous infection control. COVID-19, however, has resulted in a global pandemic, overwhelming health care systems worldwide and resulting in millions of deaths globally.

Although rapid development and approval of vaccines have helped control the infectious COVID-19 pandemic significantly, there is an urgent need for novel antiviral drugs that specifically target the COVID-19 causing coronavirus SARS-CoV-2 for the treatment of COVID-19. Alongside vaccines, antiviral therapeutics are an important part of combating the ongoing and unprecedented COVID-19 pandemic triggered by the coronavirus (SARS-CoV-2).

Several members of the family Coronaviridae constantly circulate in the human population and usually cause mild respiratory disease (Corman et al., 2019, Internist (Berl.), 2019, 60, 1136-1145). In contrast, the severe acute respiratory syndrome coronavirus (SARS-CoV) and the Middle East respiratory syndrome coronavirus (MERS-CoV) are transmitted from animals to humans and cause severe respiratory diseases in afflicted individuals, SARS and MERS, respectively (Fehr et al., Annu. Rev. Med., 2017, 68, 387-399). SARS emerged in 2002 in Guangdong province, China, and its subsequent global spread was associated with 8,096 cases and 774 deaths (de Wit et al., Nat. Rev. Microbiol., 2016, 14, 523-534). Chinese horseshoe bats serve as natural reservoir hosts for SARS-CoV (Lau et al., Proc. Natl. Acad. Sci. USA, 2005, 102, 14040-14045); Li et al., Science, 2005, 309, 1864-1868). Human transmission was facilitated by intermediate hosts like civet cats and raccoon dogs, which are frequently sold as food sources in Chinese wet markets (Guan et al., Science, 2003, 302, 276-278). There were no vaccines or drugs available to combat SARS in 2002-03 and the SARS pandemic in 2002 and 2003 was finally stopped by conventional control measures, including travel restrictions and patient isolation.

In December 2019, a new infectious respiratory disease emerged in Wuhan, Hubei province, China (Huang et al., Lancet, China, 2020; Wang et al., Lancet, 2020, 395, 470-473; Zhu et al., N Engl J Med., 2020, 382, 727-733. DOI: 10.1056/NEJMoa2001017; Q. Li, et al. N. Engl. J. Med. 2020, 382(13), 1199. DOI: 10.1056/NEJMoa2001316). An initial cluster of infections was linked to Huanan seafood market, potentially due to animal contact. Subsequently, human-to-human transmission occurred (Chan et al., Lancet, 2020,395, 514-523) and the disease, now termed coronavirus disease 19 (COVID-19) rapidly spread within China and then to South Korea, Iran, Italy, Spain, U.K., USA and virtually the whole world. A novel coronavirus, SARS-coronavirus 2 (SARS-CoV-2), which is closely related to SARS-CoV, was detected in patients and is believed to be the etiologic agent of the new lung disease (Zhu et al., N Engl J Med., 2020, 382, 727-733). On Feb. 12, 2020, a total of 44,730 laboratory-confirmed infections were reported in China, including 8,204 severe cases and 1,114 deaths (WHO, 2020). Infections had spread rapidly due to international travel and continue to increase in numbers throughout the world, particularly China, South Korea, Italy, Spain, Iran, UK, and USA resulting in the unprecedented viral disease, Covid-19, which soon after was declared pandemic worldwide.

Genome sequencing revealed that the responsible pathogen was a coronavirus with 80% nucleotide sequence identity to the severe acute respiratory syndrome coronavirus (SARS-CoV) responsible for the 2002-2004 outbreak originating from Foshan, Guangdong province, China (T. G. Ksiazek, et al. N. Engl. J. Med. 2003, 348(20), 1953. DOI: 10.1056/NEJMoa030781; C. Drosten, et al. N. Engl. J. Med. 2003, 348(20), 1967. DOI: 10.1056/NEJMoa030747; R. Lu, et al. Lancet. 2020, 395, 565. DOI: 10.1016/50140-6736(20) 30251-8.; P. Zhou, et al. Nature. 2020, 579, 270. DOI: 10.1038/s41586-020-2012-7). This new coronavirus was named ‘severe acute respiratory syndrome coronavirus 2 (SARS-CoV-2) by the International Committee on Taxonomy of Viruses (Coronaviridae Study Group of the International Committee on Taxonomy of Viruses, Nat. Microbiol. 2020, 5, 536). The disease was called ‘coronavirus disease 2019 (COVID-19) by the World Health Organization (WHO). The virus has since spread globally, infecting over half a billion people and causing over nearly 6.3 million deaths as of June 2022 (“World Health Organization Coronavirus (COVID-19) Dashboard” can be found under https://covid19.who.int.).

The coronavirus 3-chymotrypsin-like protease (3CL^{Pro}; also known as ‘main protease’ or M^{Pro}, or nsp5 protease) is deemed an attractive drug target due to its critical role in coronavirus polyprotein processing. It cleaves the polyprotein at more than 11 sites to yield essential proteins required for virus replication and pathogenesis (R. Banerjee, et al. Drug Discovery Today 2021, 26(3), 804. DOI: 10.1016/j.drudis.2020.12.005; W. Xiong, et al. Med. Res. Rev. 2021, 41, 1965. DOI: 10.1002/med.21783; H. Yang, et al. RSC Med. Chem. 2021, 12, 1026. DOI: 10.1039/d1md00066g; Z. Jin, et al. Nature. 2020, 582, 289. DOI: 10.1038/s41586-020-2223-y; S. Ullrich, et al., Bioorg. Med. Chem. Lett. 2020, 30, 127377. DOI: 10.1016/j.bmcl.2020.127377). As 3CL^{Pro} has no reported human homologues, a 3CL^{Pro} inhibitor should not inhibit any human proteases and hence, reduce the risk of any side-effects. 3CL^{Pro} is a cysteine protease with a unique substrate preference for a P1 glutamine, hydrolyzing the peptide bond at the C-terminus of glutamine. Particularly noteworthy is that the 3CL^{Pro} of SARS-CoV and SARS-CoV-2 share 96% amino acid sequence identity so inhibitors designed for the former should work equally well on the latter (R. Banerjee, L. et al. Drug Discovery Today 2021, 26(3), 804. DOI: 10.1016/j.drudis.2020.12.005; A. K. Ghosh, et al. ChemMedChem. 2020, 15, 907. DOI: 10.1002/cmdc.202000223).

Severe acute respiratory syndrome coronavirus 2 (SARS-CoV-2), a novel coronavirus, is the causative agent of coronavirus disease 2019 (COVID-19). The worldwide outbreak of COVID-19 caused by severe acute respiratory syndrome coronavirus 2 (SARS-CoV-2) has become a global pandemic. Alongside vaccines, antiviral therapeutics are an important part of the healthcare response to countering the ongoing threat presented by COVID-19. Although a few vaccines and antiviral small molecule drugs (Remdesivir, Paxolvid (Nirmatrelvir+Ritonavir), molnupiravir), and monoclonal antibodies (Casirivimab+Imdevimab and Sotrovimab), (https://www.fda.gov/consumers/consumer-updates/know-your-treatment-options-covid-19) have so far been approved or granted emergency use authorization by the United States Food and Drug Administration there still is an urgent need for effective antiviral drugs.

The U.S. FDA has issued an Emergency Use Authorization (EMA) of Paxlovid™ (combination of Nirmatrelvir+Ritonavir tablets) for the treatment of mild-to-moderate coronavirus disease 2019 (COVID-19) in adults and pediatric patients (12 years of age and older weighing at least 40 kilograms or about 88 pounds) with positive results of direct SARS-CoV-2 testing, and who are at high risk for progression to severe COVID-19, including hospitalization or death. This US FDA authorization of Nirmatrelvir as an oral pill treatment for COVID-19 is a major step forward in the fight against the COVID-19 global pandemic.

Nirmatrelvir, a SARS (CoV-2) antiviral drug, is a new tool to combat COVID-19 at a crucial time in the pandemic as new variants continue to emerge. The emergency use of authorization (EUA) of Nirmatrelvir as an oral drug makes antiviral treatment more accessible to patients who are at high risk for progression to severe COVID-19.

Normatrelvir is the active pharmaceutical ingredient of the oral antiviral drug, Paxlovid. Nirmatrelvir, is a potent and selective SARS-CoV-2 main protease (M^{Pro}) inhibitor. SARS-CoV-2 main protease (M^{Pro}) is also referred to as 3CL or nsp5 protease. The COVID-19 drug Paxlovid consists of Nirmatrelvir and Ritonavir. Ritonavir is a HIV-1 protease inhibitor and also a CYP3A inhibitor acts as a Nirmatrelvir booster that reduces CYP 3A4 induced metabolic degradation of the drug. Nirmatrelvir is one of the most effective antiviral drugs for the treatment of COVID-19, to date. Nirmatrelvir is an orally active SARS-CoV-2 main protease inhibitor (M^{Pro}) with in vitro pan-human coronavirus antiviral activity. It has been reported that Nirmatrelvir has demonstrated its antiviral activity against all coronaviruses that are known to infect humans (Owen D R, Allerton C M N, Anderson A S, et al. An oral SARS-CoV-2 M^{PRO} inhibitor clinical candidate for the treatment of COVID-19. Science. 2021; 374 (6575): 1586-1593. Available at: https://www.ncbi.nlm.nih.gov/pubmed/34726479).

Nirmatrelvir is an orally bioavailable main protease (M^{Pro}) inhibitor that is active against the SARS-CoV-2 main protease M^{PRO} that plays an essential role in viral replication by cleaving the 2 viral polyproteins (Pillaiyar T, et al., An overview of severe acute respiratory syndrome-coronavirus (SARS-CoV) 3CL protease inhibitors: peptidomimetics and small molecule chemotherapy. J. Med. Chem. 2016;59(14), 6595-6628; Available at: https://www.ncbi.nlm.nih.gov/pubmed/26878082).

PAXLOVID is nirmatrelvir tablets co-packaged with ritonavir tablets (2:1). Nirmatrelvir inhibits a SARS-CoV-2 proteinase to prevent the virus from replicating, and ritonavir slows down nirmatrelvir's enzymatic (cytochrome P450, CYP3A) metabolic degradation to help it remain in the body for a longer duration of action at higher concentrations. Paxlovid is administered as three tablets (two tablets of nirmatrelvir and one tablet of ritonavir) taken together orally twice daily for five days, for a total of 30 tablets. Paxlovid is not authorized for use for longer than five consecutive days. Nirmatrelvir is packaged with ritonavir, a strong cytochrome P450 (CYP) 3A4 inhibitor and pharmacokinetic boosting agent that has been used to boost HIV protease inhibitors. Co-administration of ritonavir is required to increase nirmatrelvir concentrations to the target therapeutic range.

Because Paxlovid (Nirmatrelvir+Ritonavir) works, in part, by inhibiting a group of enzymes (Cytochrome P450 or CYP) that break down certain drugs, Paxlovid is contraindicated with certain drugs that are highly dependent on those CYP enzymes for metabolism and for which elevated concentrations of certain drugs are associated with serious and/or life-threatening reactions. Paxlovid is also contraindicated with drugs that, conversely, strongly induce those same enzymes, leading to the faster breakdown of nirmatrelvir or ritonavir, as reduced concentrations of nirmatrelvir or ritonavir may be associated with potentially losing virologic response and developing viral resistance. Paxlovid cannot be started immediately after discontinuing such medications because the effects of those medications remain after discontinuation. A complete list of drugs that should not be taken in combination with Paxlovid, sis available in the fact sheet for healthcare providers.

Because nirmatrelvir is co-administered with ritonavir, there are a significantly large number of contraindications attributable in large part to ritonavir, which is an inhibitor of cytochrome P450 enzyme 3A4 (CYP3A4), the enzyme typically responsible for drug metabolism. Ritonavir causes adverse effects and increases pill burden for patients. Ritonavir use is also responsible for several serious contraindications that include antihypertensive drugs (calcium channel blocker-amlodipine, diltiazem, felodipine, nicardipine, nifedipine), cardiac glycoside (digoxin), endothelin receptor antagonist (bosentan), ergot derivatives (dihydroergotamine, ergotamine, methylergonovine), Hepatitis C direct acting antivirals (elbasvir/grazoprevir, glecaprevir/pibrentasvir, ombitasvir/paritaprevir/ritonavir and dasabuvir, sofosbuvir/velpatasvir/voxilaprevir), HMG-CoA reductase inhibitors (lovastatin, simvastatin, atorvastatin, rosuvastatin), Hormonal contraceptive (ethinyl estradiol), Immunosuppressants (cyclosporine, tacrolimus, sirolimus), Long-acting beta-adrenoceptor agonist (salmeterol), Narcotic analgesics (fentanyl, methadone), PDE5 inhibitor (sildenafil (Revatio®, when used for pulmonary arterial hypertension), Sedative/hypnotics (triazolam,oral and parenterally administered midazolam, midazolam), Systemic corticosteroids (betamethasone, budesonide, ciclesonide, dexamethasone, fluticasone, methylprednisolone, mometasone, prednisone, triamcinolone). Paxlovid (combination of two 150 mg tablets of Nirmatrelvie and one 100 mg tablet of ritonavir) is not recommended in patients with severe renal impairment (eGFR<30 mL/min based on CKD-EPI formula) until more data are available; the appropriate dosage for patients with severe renal impairment has not been determined.

Paxlovid (Nirmatrelvir+ritonavir) is contraindicated with drugs that are highly dependent on CYP3A for clearance and for which elevated concentrations are associated with serious and/or life-threatening reactions specifically, alphal-adrenoreceptor antagonist alfuzosin, analgesics pethidine and propoxyphene, antianginal ranolazine, antiarrhythmic amiodarone, dronedarone, flecainide, propafenone and quinidine, anti-gout colchicine, antipsychotics lurasidone, pimozide, and clozapine, ergot derivatives dihydroergotamine, ergotamine and methylergonovine, HMG-CoA reductase inhibitors lovastatin and simvastatin, PDE5 inhibitors sildenafil (Revatio®) when used for pulmonary arterial hypertension, Sedative/hypnotics triazolam and oral midazolam.

Paxlovid (Nirmatrelvir+ritonavir) is contraindicated with drugs that are potent CYP3A inducers where significantly reduced nirmatrelvir or ritonavir plasma concentrations may be associated with the potential for loss of virologic response and possible resistance. Paxlovid (Nirmatrelvir+ritonavir) cannot be started immediately after discontinuation of any of the following medications due to the delayed offset of the recently discontinued CYP3A inducer, anticancer drug apalutamide, anticonvulsant carbamazepine, phenobarbital and phenytoin, atimycobacterial rifampin, and herbal products St. John's wort (hypericum perforatum).

This invention is concerned with disclosing novel deuterated antiviral drugs that will eliminate contraindications associated with the therapeutic use of Nirmatrelvir discussed above. The contraindications discussed above are in large part due to the use of the CYP3A inhibitor ritonavir. The novel deuterated compounds of the present of the general formula I eliminate the use of the CYP3A inhibitor ritonavir as the Nirmatrelvir booster. Elimination of ritonavir from the combination (Nirmatrelvir+ritonavir) by the deuterated compounds of the present invention will help treatment of all the patients that cannot be treated with Paxlovid (2 normatrelvir+1 ritonavir) for example patients with severe renal impairment patients (eGFR<30 mL/min); Paxlovid is not recommended for these kidney disease patients. Reduced dose of Paxlovid (Nirmatrelvir) is recommended for patients with moderate renal impairment (eGFR>30 to <60 mL/min).

In general, combining drugs with cytochrome P450 (CYP) inhibitors is not a safe efficient strategy for decreasing drug metabolism and clearance. The inhibition of activity of a CYP enzyme can affect the metabolism and clearance of other drugs metabolized by that same enzyme. CYP inhibition can cause other drugs to accumulate in the body to toxic levels.

A potentially robust, satisfactory and efficacious strategy for improving metabolism of a drug (pharmacokinetic characteristics of a drug) is by modification of the chemical structure of a drug by substitution of hydrogen (¹H) atom/s, that are susceptible (prone) to the oxidative degradation (hydroxylation in most cases) by cytochrome P450 (CYP) enzymes, by deuterium (D or ²H) atom/s. This innovative approach of deuterium substitution for hydrogen atoms of a drug molecule, that are vulnerable to CYP-mediated metabolism, is designed to reduce the deleterious metabolic degradation and decease in efficacy of a drug. This method of deuterium substitution of hydrogen atom or atoms in a drug compound can also reduce or completely eliminate the formation of undesirable and potentially toxic metabolites. Deuterium (D or ²H)) is a safe, stable, non-radioactive isotope of hydrogen. Compared to hydrogen (H), deuterium (D) forms stronger bonds with carbon. This increase in bond strength of the carbon-deuterium bond imparted by deuterium can positively impact the ADME (Absorption, Distribution, Metabolism, Excretion) properties of a drug. This enhancement in ADME characteristics of a drug molecule significantly increases the potential for improving the efficacy, safety, and/or tolerability of a drug. Concurrently, since the atomic size and shape of deuterium are essentially identical to those of hydrogen, replacement of hydrogen by deuterium would not be expected to change the biochemical activity or potency and selectivity of the drug (deuterated drug or D-Drug) as compared to the original drug molecule (hydrogen containing drug or H-Drug).

During the last four decades, effects of deuterium substitution on drug metabolism have been reported only on a small number of drugs (Blake, M I et al. J. Pharm Sci, 1975, 64: 367-91; Foster, A B et al. Adv Drug Res, 1985, 14:1-40; Kushner, D J et al., Can J Physiol Phatmacol, 1999, 79-88; Fischer, M B et al. Curr Opin Drug Discov Devol, 2006, 9: 101-109). The results have been variable and unpredictable. While for some compounds deuterium substitution caused decreased in vivo metabolic clearance but for others there was no change in metabolism. Still others demonstrated increase in metabolic clearance. The variability in deuterium effects has also led experts to question or dismiss drug modification by deuterium substitution of hydrogen as a viable drug design strategy for inhibiting adverse metabolism (Foster at p 35 and Fischer at p 101).

The effects of deuterium modification on the metabolic pathways or metabolic properties of a drug are not predictable even when deuterium atoms are incorporated at known metabolic sites. Only by actually preparing and testing a deuterated drug can one determine if and how the rate of metabolism will differ from that of its non-deuterated counterpart. See for example Fukuto et al (J. Med. Chem. 1991, 34, 2871-76). Many drugs contain multiple sites where CYP-mediated metabolism can occur. The site(s) where deuterium-substitution is required and the extent of deuteration necessary to see an effect on metabolism, if any, will be different for each drug.

Nirmatrelvir is a SARS-CoV-2 main protease (Mpro: also referred to as 3CLpro or nsp5 protease) inhibitor for the treatment of mild-to-moderate coronavirus disease 2019 (COVID-19) in adults and pediatric patients (12 years of age and older weighing at least 40 kg) with positive results of direct severe acute respiratory syndrome coronavirus 2 (SARS-CoV-2) viral testing, and who are at high risk for progression to severe COVID-19, including hospitalization or death.

The chemical name of Nirmatrelvir, the active ingredient of the antiviral drug Paxlovid is (1R,2S,5S)-N-((1S)-1-Cyano-2-((3S)-2-oxopyrrolidin-3-yl)ethyl)-3-((2S)-3,3-dimethyl-2-(2,2,2-trifluoroacetamido)butanoyl)-6,6-dimethyl-3-azabicyclo[3.1.0]hexane-2-carboxamide]. It has a molecular formula of C23H32F3N5O4 and a molecular mass (weight) of 499.54. Nirmatrelvir has the following structural formula:

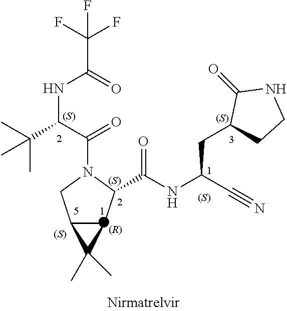

Despite the beneficial therapeutic use of the drug Nirmatrelvir, there is a continuing need for new safer and more effective small molecule oral therapeutics for the treatment of SARS-CoV-2 (COVID-19) disease and new diseases that can be caused by new variants.

### DETAILED DESCRIPTION OF THE INVENTION

The present invention is concerned with novel deuterium-enriched compounds of the general structural formula I, their enantiomers and diastereoisomers, and pharmaceutical salts, and compositions thereof,

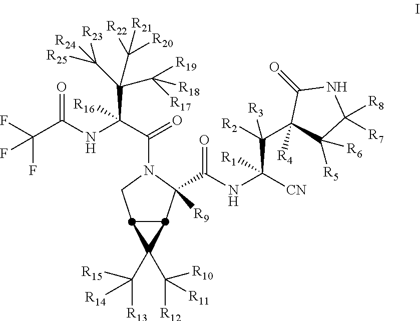

Wherein,
R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅ are independently D (deuterium), H (hydrogen).

Another embodiment encompasses the deuterium enriched compounds of formula I wherein R₇ and R₈ are deuterium and at least one of R₁₆, R₁₇, R₁₈, R₁₉, R₂₀, R₂₁, R₂₂, R₂₃, R₂₄, R₂₅ is deuterium.

Another embodiment encompasses the deuterium enriched compounds of formula I wherein R₇ and R₈ are deuterium and at least one of R₉, R₁₀, R₁₁, R₁₂, R₁₃, R₁₄, R₁₅, is deuterium.

Another embodiment encompasses the deuterium enriched compounds of formula I wherein R₇ and R₈ are deuterium and at least one of R₁, R₂, R₃, R₄, R₅, R₆, is deuterium.

The compounds of the invention can also be used with other Covid-19 treatments, examples of such treatments would include remdesivir, molnupiravir and mono-clonal antibody cocktails. Examples of the mono-clonal antibody cocktails include bamlanivimab, bebtelovimab, casirivimab, cilgavimab, etesevimab, imdevimab, sotrovimab, and tixagevimab.

These mono-clonal antibody cocktails will change as new variants of the virus arise.

The compounds of the invention can also be used with angiotensin II receptor antagonist; examples of useful angiotensin II receptor antagonists include Losartan, Valsartan, Candesartan, Irbesartan, Olemsartan, Telmisartan and Eprosartan.

The compounds of the invention can also be used with angiotensin converting enzyme (ACE) inhibitors; examples of useful angiotensin converting enzyme inhibitor.include Enalapril, Lisinopril, Captopril, Benazepril, Fosinopril, Ramipril, Quinapril, and Perindopril.

The compounds of the invention can also be used with angiotensin converting enzyme-2 (ACE-2) inhibitors; examples of potentially useful angiotensin converting enzyme-2 inhibitor include
((S)-1-carboxy-2-(1-(3,5-dichlorobenzyl)-1H-imidazol-5-yl)ethyl)leucine-d₉

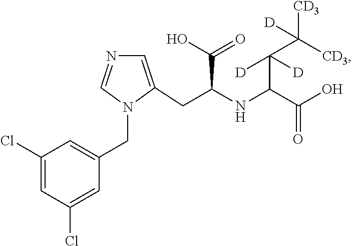 [SMILES: [2H]C([2H])([2H])C([2H])(C([2H])([2H])[2H])C([2H])([2H])C(N[C@@H](Cc1cncn1Cc1cc(Cl)cc(Cl)c1)C(=O)O)C(=O)O]

((S)-1-carboxy-2-(1-(3,5-dichlorobenzyl)-1H-imidazol-5-yl)ethyl)-L-leucine-d₉

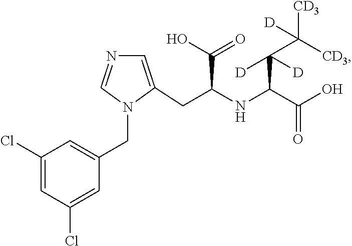 [SMILES: [2H]C([2H])([2H])C([2H])(C([2H])([2H])[2H])C([2H])([2H])[C@H](N[C@@H](Cc1cncn1Cc1cc(Cl)cc(Cl)c1)C(=O)O)C(=O)O]

and
((S)-1-carboxy-2-(1-(3,5-dichlorobenzyl)-1H-imidazol-5-yl)ethyl)-D-leucine-d₉

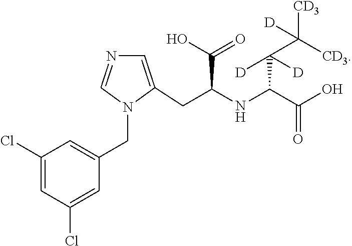 [SMILES: [2H]C([2H])([2H])C([2H])(C([2H])([2H])[2H])C([2H])([2H])[C@@H](N[C@@H](Cc1cncn1Cc1cc(Cl)cc(Cl)c1)C(=O)O)C(=O)O]

The compounds of the invention can also be used with calcium channel antagonists; examples of useful calcium channel antagonists include amlodipine, felodipine, Isradipine, Nicardipine, Nifedipine, Nimodipine and Nitrendipine.

Compound of Formula I are novel deuterium containing analog compounds of the COVID-19 oral drug Nirmatrelvir. These compounds have high potency (low nm IC₅₀) selectivity for inhibiting Severe Acute Respiratory Syndrome Coronavirus-2 (SARS-CoV-2) or Severe Acute Respiratory Syndrome Coronavirus-2 main protease (SARS-CoV-2 M^{Pro}), and therefore are useful in the treatment of the unprecedented pandemic disease Covid-19 caused by the Severe Acute Respiratory Syndrome-Coronavirus 2 (SARS-CoV-2). The compounds of this invention are also useful for the treatment of Severe Acute Respiratory Syndrome-Coronavirus (SARS-CoV), Middle East Respiratory Syndrome Coronavirus (MERS-CoV), and potential variants of SARS(Cov)-2 virus.

Deuterium (D or ²H) is a stable non-radioactive isotope of hydrogen (H) and has an atomic weight of 2.0144. Hydrogen occurs naturally as a mixture of the isotopes ¹H, D (²H or deuterium), and T (³H or tritium) and the natural abundance of deuterium is 0-015%. One of ordinary skill in the art recognizes that in all compounds containing H atom, H actually represents a mixture of H and D, with about 0-015% of D. So therefore, compounds with a level of D (deuterium) that has been enriched to be greater than its natural abundance of 0.015%, should be considered unnatural and as a result novel as compared to their corresponding non-enriched counterparts.

The carbon-hydrogen bonds contain a naturally occurring distribution of hydrogen isotopes, namely ¹H or protium (about 99.9844%), ²H or deuterium (D) (about 0.0156%), and ³H or tritium (in the range between about 0.5 and 67 tritium atoms per 1018 protium atoms). Higher levels of deuterium incorporation produce a detectable Kinetic Isotope Effect [Werstiuk, N. H.; Dhanoa, D. S.; Timmins, G. Can J. Chem. 1979, 57, 2885; Werstiuk, N. H.; Dhanoa, D. S.; Timmins, G. Can J. Chem. 1983, 61, 2403], that could improve the pharmacokinetic, pharmacologic and/or toxicologic parameters of compounds of formula I in comparison to compounds having naturally occurring levels of deuterium and their corresponding hydrogen (protium) analogs.

Suitable modifications of certain carbon-hydrogen (C—H) bonds into carbon-deuterium (C-D) bonds may generate novel substituted compounds of structural formula I with unexpected and non-obvious improvements of pharmacological, pharmacokinetic and toxicological properties in comparison to the non-isotopically enriched compounds. This invention relies on the judicious and successful application of chemical kinetics to drug design. Deuterium incorporation levels in the compounds of the invention are significantly higher than the naturally-occurring levels and are sufficient to induce at least one substantial improvement as described herein.

“Deuterium enrichment” refers to the percentage of incorporation of deuterium at a given site on the molecule instead of a hydrogen atom. For example, deuterium enrichment of 1% means that in 1% of molecules in a given sample a particular site is occupied by deuterium. Because the naturally occurring distribution of deuterium is about 0.0156%, deuterium enrichment in compounds synthesized using non-enriched starting materials is about 0.0156%.

It can be a significant synthetic challenge to produce 100% deuterium at a specific site of a compound. High levels of deuterium content in a compound can be produced either by Hydrogen-Deuterium (H-D) exchange or by synthesizing the compound for specific deuteration. The H-D exchange is readily achieved in case of H atoms attached to heteroatoms for example in cases of carboxylic acids (COOH), sulfonamides (SO2NH₂, CONHSO₂-aryl, CONHSO₂-alkyl), alcohols (OH), basic amines (NH₂), etc. However, these incorporated deuterium (D) attached to heteroatoms (O, N, S) readily revert back to hydrogen (H) upon exposure to water or any acidic compounds containing H atoms. The preferred deuterium containing compounds contain deuterium (D) directly attached to carbon atoms of the structure of the compounds of this present invention.

In some embodiments, the deuterium enrichment in the compounds of the present invention is greater than 4%, 5%, 6%, 7%, 8%, 9% or 10%. In other embodiments, the deuterium enrichment in the compounds of the present invention is greater than 20%. In further embodiments, the deuterium enrichment in the compounds of the present invention is greater than 50%. In some embodiments, the deuterium enrichment in the compounds of the present invention is greater than 70%. In some embodiments, the deuterium enrichment in the compounds of the present invention is greater than 90%.

This invention is concerned with deuterium-enriched compounds of the general structural formula I, their enantiomers, diastereomers, pharmaceutical acceptable salts thereof,

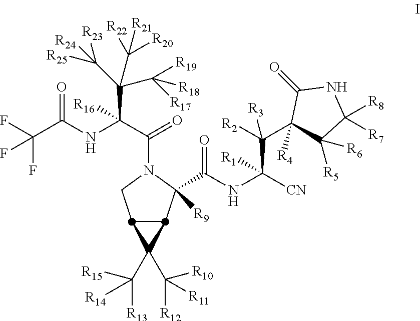

Wherein,
R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅ are independently D (deuterium), H (hydrogen);

Wherein D is deuterium atom present in the compounds of formula I and about 1%-100% enrichment of deuterium is incorporated.

The reaction scheme conceptualized and used for the synthesis of compounds and intermediates of this invention are general. It will be understood by those skilled in the art of organic synthesis that one or more functional groups present in a given compound of the invention may render the molecule incompatible with a particular synthetic sequence. In such a case an alternative synthetic route, an altered order of steps or a strategy of protection and deprotection may be employed. The reactions are performed in a solvent appropriate to the reagents and materials employed and suitable for the transformation being effected. It is understood by those skilled in the art of organic synthesis that the functionality present on the reactants and reagents being employed should be consistent with the chemical transformations being conducted. Depending upon the reactions and techniques to be used optimal yields may require changing the order of synthetic steps or use of protecting groups followed by deprotection. In all cases the particular reaction conditions, including reagents, solvent, temperature and time, should be chosen so that they are consistent with the nature of the functionality present in the molecule.

The compounds useful in the novel method treatment of this invention may form salts with various inorganic and organic acids and bases, which are also within the scope of the invention. Such salts include alkali metal salts like sodium and potassium salts, ammonium salts, alkaline earth metal salts such as calcium and magnesium salts, salts with organic bases for example dicyclohexylamine salts, N-methyl-D-glucamine salts, salts with amino acids e.g., arginine, lysine, etc. In addition, salts with organic and inorganic acids may be prepared; e.g., HCl, HBr, H₂SO₄, H₃PO₄, methanesulfonic, toluenesulfonic, maleic, fumaric, camphorsulfonic acid.

The salts can be formed by conventional means, such as by reacting the free acid or free base forms of the product with one or more equivalents of the appropriate base or acid in a solvent or medium in which the salt is insoluble, or in a solvent such as water, which is then removed in vacuo or by freeze-drying or by exchanging the cations of an existing salt for another cation on a suitable ion exchange resin.

The compounds useful in the novel method treatment of this invention form solvates with various solvents, which are also within the scope of the invention. Such solvates include methyl tert-butyl ether solvates, diethyl ether or others.

It will be appreciated that the compounds of general Formula I in this invention may be derivatized at functional groups to provide prodrug derivatives, which are capable of conversion back to the parent compounds in vivo. The concept of prodrug administration has been extensively reviewed (e.g. A. A. Sinkula in Annual Reports in Medicinal Chemistry, Vol 10, R. V. Heinzelmann, ED., Academic Press, New York, London, 1975, Ch 13, pp 306-326; H. Ferres, Drugs of Today, Vol 19, 499-538, 1983, and J. Med. Chem.,18, 172, 1975). Examples of such prodrugs include the physiologically acceptable and metabolically labile ester derivative, such as lower alkyl (e.g. methyl or ethyl esters), aryl (e.g. 5-indanyl esters), alkenyl (e.g. vinyl esters), alkoxyalkyl (e.g. methoxymethyl esters), alkylthioalkyl (e.g. methylthiomethyl esters), alkanoyloxyalkyl (e.g. pivaloyloxymethyl esters), and substituted or unsubstituted aminomethyl esters (e.g. 2-dimethylaminoethyl esters). Additionally, any physiologically acceptable equivalents of the compounds of general structural formula I, similar to the metabolically labile esters, which are capable of producing the parent compounds of general Formula I in vivo, are within the scope of this invention.

The compounds of the present invention exhibit poor water solubility. The invention is intended to enhance the oral bioavailability of the compounds of formula I by using liposome technologies (Lee, M-K., et al. The compounds of the present invention exhibit poor water solubility (water-insoluble compounds). The invention is intended to enhance the oral bioavailability of the compounds of formula I by using liposome technologies (Lee, M-K., et al. Liposomes for Enhanced Bioavailability of Water-Insoluble Drugs: In Vivo Evidence and Recent Approaches, Pharmaceutics 2020, 12, 264; doi:10.3390/pharmaceutics12030264).

It will be further appreciated that the majority of compounds of general Formula I claimed herein are asymmetric and that both the racemic compounds and the individual non-racemic chiral enantiomers are considered to be within the scope of this invention. The compounds of the present invention may have various isomers including all stereoisomers of asymmetric atoms (enantiomers and diastereomers) and geometric, tautomeric or rotamers, and all isomers are considered to be part of the present invention. All processes used to prepare compounds of the present invention and intermediates made therein are considered to be part of the present invention. The racemic compounds of this invention may be resolved to provide individual enantiomers utilizing methods known to those skilled in the art of organic synthesis. diastereoisomers of the compounds of general Formula I, their pharmaceutically acceptable salts and their prodrug forms are also included within the scope of this invention.

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder.

The combination of compounds is preferably a synergistic combination. “Pharmaceutically acceptable salts” refer to derivatives of the disclosed compounds wherein the parent compound is modified by making acid or base salts thereof. Examples of the pharmaceutically acceptable salts include, but not limited to, mineral or organic acid salts of the basic residues. The pharmaceutically acceptable salts include but not limited to HCl, HBr, HI, potassium (K), sodium (Na), calcium (Ca), magnesium (Mg), acetic, trifluoroacetic, citric, ascorbic, benzoin, methanesulfonic (mesylate), benzenesulfonic, bicarbonic, carbonic, ethane disulfonic, edetic, fumaric, maleic, lactic, malic, mandelic, gluconic, glutamic, glycolic, glycollyarsanilic, lauryl, hexylresorcinic, hyrdabamic, hydroxymaleic, hydroxynaphthoic, isethionic, lactobionic, napsylic, nitric, oxalic, pamoic, pantothenic, phenyllacetic, phosphoric, polygalacturonic, propionic, salicyclic, stearic, subacetic, succinic, sulfamic, sulfanilic, sulfuric, tannic, tartaric, tolouenesulfonic, and p-bromobenzenesulfonic.

### Definitions

For purposes of the present invention, as described and claimed herein, the following terms are defined as follows:

As used herein, the terms “comprising” and “including” are used in their open, non-limiting sense. The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups, which may be present in the compounds of formula I. The compounds of formula I that are basic in nature are capable of forming a wide variety of salts with various inorganic and organic acids. The acids that may be used to prepare pharmaceutically acceptable acid addition salts of such basic compounds of formula I are those that form non-toxic acid addition salts, i.e. salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

Certain compounds of formula I may have asymmetric centers and therefore exist in different enantiomeric forms. All optical isomers and stereoisomers of the compounds of formula I and mixtures thereof, are considered to be within the scope of the invention. With respect to the compounds of formula I the invention includes the use of a racemate, one or more enantiomeric forms, one or more diastereomeric forms, or mixtures thereof. The compounds of formula I may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

This invention also encompasses pharmaceutical compositions containing and methods of treating SARS infections through administering prodrugs of compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula I. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The compounds of the invention can also be used in combination with other drugs. For example, dosing a SARS coronavirus infected patient with the SARS coronavirus 3CL protease (or M^{Pro}) inhibitor of the invention and an interferon, such as interferon alpha, or a pegylated interferon, such as PEG-lntron or Pegasus, may provide a greater clinical benefit than dosing either the interferon, pegylated interferon or the SARS coronavirus inhibitor alone. Examples of greater clinical benefits could include a larger reduction in symptoms, a faster time to alleviation of symptoms, reduced lung pathology, a larger reduction in the amount of SARS coronavirus inthe patient (viral load), and decreased mortality.

The term “interfering with or preventing” SARS-related coronavirus (“SARS”) viral replication in a cell means to reduce SARS replication or production of SARS components necessary for progeny virus in a cell as compared to a cell not being transiently or stably transduced with the ribozyme or a vector encoding the ribozyme. Simple and convenient assays to determine if SARS viral replication has been reduced include an ELISA assay for the presence, absence, or reduced presence of anti-SARS antibodies in the blood of the subject (Nasoff et al., PNAS 88:5462-5466, 1991), RT-PCR (Yu et al., in Viral Hepatitis and Liver Disease 574-477, Nishioka, Suzuki and Mishiro (Eds.); Springer-Verlag Tokyo, 1994). Such methods are well known to those of ordinary skill in the art. Alternatively, total RNA from transduced and infected “control” cells can be isolated and subjected to analysis by dot blot or northern blot and probed with SARS specific DNA to determine if SARS replication is reduced. Alternatively, reduction of SARS protein expression can also be used as an indicator of inhibition of SARS replication. A greater than fifty percent reduction in SARS replication as compared to control cells typically quantitates a prevention of SARS replication. If an inhibitor compound used in the method of the invention is a base, a desired salt may be prepared by any suitable method known to the art, including treatment of the free base with an inorganic acid (such as hydrochloric acid, hydrobromic acid, sulfuric acid, nitric acid phosphoric acid, and the like), or with an organic acid (such as acetic acid, maleic acid, succinic acid, mandelic acid, fumaric acid, malonic acid, pyruvic acid, oxalic acid, glycolic acid, salicylic acid, pyranosidyl acid (such as glucuronic acid or galacturonic acid), alpha-hydroxy acid (such as citric acid or tartaric acid), amino acid (such as aspartic acid or glutamic acid), aromatic acid (such as benzoic acid or cinnamic acid), sulfonic acid (such as p-toluenesulfonic acid or ethanesulfonic acid}, and the like. In the case of inhibitor compounds, prodrugs, salts, or solvates that are solids, it is understood by those skilled in the art that the hydroxamate compound, prodrugs, salts, and solvates used in the method of the invention, may exist in different polymorph or crystal forms, all of which are intended to be within the scope of the present invention and specified formulas. In addition, the hydroxamate compound, salts, prodrugs and solvates used in the method of the invention may exist as tautomers, all of which are intended to be within the broad scope of the present invention. Solubilizing agents may also be used with the compounds of the invention to increase the compounds solubility in water or physiologically acceptable solutions. These solubilizing agents include cyclodextrans, propylene glycol, diethylacetamide, polyethylene glycol, Tween,ethanol and micelle forming agents. Oreffered solubilizing agents are cyclodextrans, particularly beta cyclodextrans and in particular hydroxypropyl betacyclodextran and sulfobutylether betacyclodextran. In some cases, the inhibitor compounds, salts, prodrugs and solvates used in the method of the invention may have chiral centers. When chiral centers are present, the hydroxamate compound, salts, prodrugs and solvates may exist as single stereoisomers, racemates, and/or mixtures of enantiomers and/or diastereomers. All such single stereoisomers, racemates, and mixtures thereof are intended to be within the broad scope of the present invention.

As generally understood by those skilled in the art, an optically pure compound is one that is enantiomerically pure. As used herein, the term “optically pure” is intended to mean a compound comprising at least a sufficient optical activity against the target to be inhibited. Preferably, an optically pure amount of a single enantiomer to yield a compound having the desired pharmacological pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. In a preferred embodiment of the present invention, “treating” or “treatment” means at least the mitigation of a disease condition in a human that is alleviated by the inhibition of the activity of one or more coronaviral 3C-like proteases, including, but not limited to the 3C-like protease of the causative agent for SARS. In the case of SARS, representative disease conditions include fever, dry cough, dyspnea, headache, hypoxemia, lymphopenia, elevated aminotransferase levels as well as viral titer. Methods of treatment for mitigation of a disease condition include the use of one or more of the compounds in the invention in any conventionally acceptable manner. According to certain preferred embodiments of the invention, the compound or compounds of the present invention are administered to a mammal, such as a human, in need thereof. Preferably, the mammal in need thereof is infected with a coronavirus such as the causative agent of SARS.

The present invention also includes prophylactic methods, comprising administering an effective amount of a compound of the invention, or a pharmaceutically acceptable salt, prodrug, pharmaceutically active metabolite, or solvate thereof to a mammal, such as a human, at risk for infection by a coronavirus. According to certain preferred embodiments, an effective amount of one or more compounds of the invention, or a pharmaceutically acceptable salt, prodrug, pharmaceutically active metabolite, or solvate thereof is administered to a human at risk for infection by the causative agent for SARS. The prophylactic methods of the invention include the use of one or more of the compounds in the invention in any conventionally acceptable manner.

### Synthesis of Compounds and Materials of the Invention

We envisioned that in order to eliminate, block or reduce the oxidative metabolic degradation of Nirmtrelvir by various isoforms of the cytochrome-450 (e.g. CYP 3A and others), we ought to substitute deuterium for hydrogen atoms that are prone (pro-metabolism) to CYP hydroxylation. In the compound structure I shown below, we substituted deuterium at most exposed alkyl groups including the gem-dimethyls (R₁₀-R₁₅ are D), tert-butyl (R₁₇-R₂₅ are D). In addition, we envisioned to incorporate deuterium (D) for hydrogen (H) at the pyrrolidinone ring moiety, specifically (R₇-R₈ are D and R₅ and R₆ are D). Furthermore, we also planned to inhibit the potential deleterious metabolic switching, so the deuteration at the methylene (R₂ and R₃ are D) and the alpha-hydrogen atoms at the three asymmetric amino acid carbon centers, (R₁, R₉ and R₁₆ are D) was envisioned.

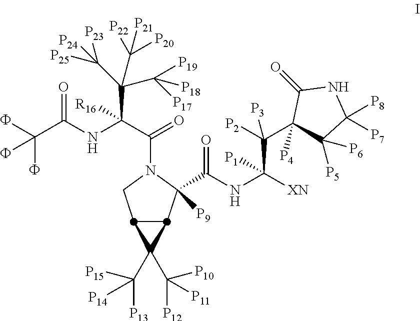

Preparation of compounds of structural formula I described below are general. It should be understood by those skilled in the art of chemical synthesis that some functional groups may not be compatible for certain synthetic routes and those cases may require appropriate changes including alternative starting materials, building blocks, intermediates, synthesis, synthetic methods process, appropriate sequence of synthetic steps, and compatible protection and deprotection strategy should be employed. The particular reaction conditions, such as reagents, solvents, temperature, and reaction time, should be used for conducting a synthetic reaction consistent with the nature of the functionality of the reactant and products involved.

The synthesis of the novel deuterium-enriched (or deuterium-containing) compounds of Formula I of this invention is shown below in Schemes 1-5.

Those skilled in the art will recognize or be able to ascertain using no more than routine experimentation, numerous equivalents to the specific reagents can be utilized to produce compounds of the invention. Numerous modifications and variations of the present invention are possible and therefore it is understood that within the scope of the appended claims, the invention may be practiced otherwise that as specifically described herein. Other aspects, advantages and modifications are within the scope of the invention.

### Synthesis

Reterosynthetic analysis of the compounds of the invention (I) revealed three key fragments as the building blocks (moieties) 5a-c, 13 and 21 for an efficient synthetic route to prepare the final target compounds of the invention.

The syntheses of the deuterium-containing moieties (chemical compounds) 5a-c, 13 and 21 from readily available starting materials are illustrated in Schemes 1, 2, and 3 respectively.

The synthesis of the final target compounds 28a-c from advanced intermediates 5a-c, 13 and 21 is shown in Scheme 4a-b, Scheme 4c and Scheme 5.

Alkylation of the commercially available N-Boc-L-glutamic acid dimethyl ester 1 (H, 1a and deuterated D, 1b-c) with commercially available bromoacetonitrile (or deuterated bromoacetontrile-d₂) using LiHMDS as the base gives the alkylated products 2a-c as shown in Scheme 1. Deuterated bromoacetonitrile-d₂ is prepared from alpha deuteration of the 2-bromoaceonitrile. Reduction of the nitrile group with sodium borodeuteride-d₄ (NaBD₄) and cobalt (II) chloride hexahydrate gives the cyclized oxopyrrolidinone intermediates 3ba-c. Treatment of the methyl esters 3a-c with ammonia gives the corresponding amides 4a-c. Deprotection of the t-Boc group of 4a-c with HCl in isopropanol produces advanced intermediate 5a-c.

The intermediate compound 13 (N-Boc t-butylglycine or N-Boc tert-Leucine) is synthesized fro the commercially available deuerated t-butyl chloride-d₉ 6 as shown in Scheme 2. Grignard reagent, t-butyl magnesium chloride is prepared in situ by reaction of 6 with Magnesium turnings and then it is treated with N,N-dimethyl formamide (DMF) to give the desired 2,2-dimethylpropanaldehyde-d₉ 7 (or t-butyl aldehyde-d₉).

Treatment of the aldehyde 7 with commercially available (R)-phenylglycine amide 8 under Strecker reaction conditions, sodium cyanide in acetic acid (NaCN, AcOH) affords the adduct product 9 which upon hydrolysis of nitrile moiety using sulfuric acid yields the diamide 10. The catalytic hydrogenation (H₂, Pd/C, AcOH/EtOH) of 10 gives the deuterated t-butylglycinamide-d₉ 11. Hydrolysis of the amino moiety of the bisamide 11 with HCl provides the deuterated amino acid 12 as its HCL salt. Treatment of t-butylglycine-d₉ 12 (or t-Leucine.HCl) with di-tert-butyl dicarbonate and triethylamine in dioxane produces the second key intermediate 13, N-Boc t-leucine (or t-butylglycine) as shown in Scheme 2.

The advanced intermediate 21 (Scheme 3) is synthesized from the commercially available diphenyl sulfide 14, iodoethane-d₃ (CD₃CH₂I) 15 and commercially available compound 17. Diphenyl(2,2,2-trideuteroethyl-d₃)sulfonium tetrafluoroborate 16 is synthesized by treatment of diphenyl sulfide with silver tetrafluroborate and 15. The sulfonium salt 16 is further alkylated with methyl iodide-d₃ (CD3I) to produce sulfur ylide in situ at low temperature which in turn is treated with the commercially available compound 17, (3R,7aS)-3-phenyl-1,7a-dihydropyrrolo[1,2-c]oxazol-5(3H)-one, 17 to produce the cyclic lactam compound 18, (3R,5αS,6αS,6βS)-3-Phenyl-6,6-bis(trideuteromethyl-d₃)tetrahydro-1H-cyclopropa[3,4]pyrrolo[1,2-c]oxazol-5(3H)-one (18). The reduction of 18 with Lithium aluminum hydride (LiAlH₄) gives the ring opened N-benzylated product deuterated gem-dimethyl bicyclic propyl prolinol intermediate compound 19. N-benzylated prolinol 19 is then debenzylated by its palladium-catalyzed transfer hydrogenolysis in ammonium formate yields the corresponding amino alcohol-d₆, which in turn is dissolved in dichloromethane and treated with triethylamine and di-tert-butyl dicarbonate (Boc₂O) to produce the desired bicyclic N-Boc gem-dimethyl-d₆-cyclopropyl prolinol 20. Oxidation of this amino alcohol 20 under Sharpless conditions using sodium per iodate and ruthenium trichloride direct oxidation of this amino alcohol 20 to the corresponding carboxylic acid, which upon methylation with trimethylsilyl diazomethane affords the corresponding methyl ester, which upon treatment with HCl gives the N-Boc deprotected free amino ester 21.

Synthesis of the advanced intermediate compounds 27a-c is shown in Scheme 4a-b and Scheme 4c. The deutearted gem-dimethyl cyclopropyl proline methyl ester 21 is coupled with the N-Boc tert.leucine 13 using HATU and DIEA in acetonitrile and DMF to give the coupled product 22 (Scheme 4a-b). Saponification of methyl ester of the unnatural synthesized dipeptide 22 with lithium hydroxide (LiOH) in aqueous tetrahydrofuran (THF) affords the corresponding N-Boc tert-leucinyl-(dimethyl cyclopropyl)proline 23. Deprotection of N-Boc of 23 by treatment with HCl gives the corresponding tert-leucine-dimethylcyclopropyl proline hydrochloride 24. The advanced intermediate proline derivative 24 is trifluoroacylated with ethyl trifluoroacetate in di-isopropyl ethylamine (DIEA) and methanol to afford key advanced intermediate 25. This trifluoroacetyl synthon 25 is then used to synthesize the more advanced precursors compounds to the final target deuterated Nirmatrelvir analogs.

The compound 25 is coupled with the already synthesized intermediate oxopyrrolidinone compound 5a using the EDCI and 2-hydroxypyridine in N, N-Diisopropylethylamine (DIEA) and 2-butanone to give the advanced coupled product 26a (Scheme 4a-b). The deuterated compounds 26b is synthesized from the compound 25 and 5b (Scheme 4a-b) using the same procedure as described for the synthesis of 26a. Similarly, the advanced intermediate compound 26c is synthesized by coupling of 25 and 5c using EDCI, 2-hydroxypyridine in DIEA and methanol as shown in Scheme 4c.

The advanced amide (CONH₂) intermediates 26a, 26b and 26c are individually dehydrated to the corresponding nitrile (CN) using the Burgess reagent (Methyl N-(triethylammoniosulfonyl)carbamate, inner salt) to provide the corresponding advanced compounds 27a, 27b and 27c, as MTBE solvate (t-butyl methyl ether solvates) as shown in Schemes 4a-b and Scheme 4c.

Recrystalization of MTBE solvates 27a, 27b and 27c from solvent mixture of heptane and isopropyl acetate yields the desired target final deuterated compounds of the present invention of chemical structural formula I as shown in Scheme 5.

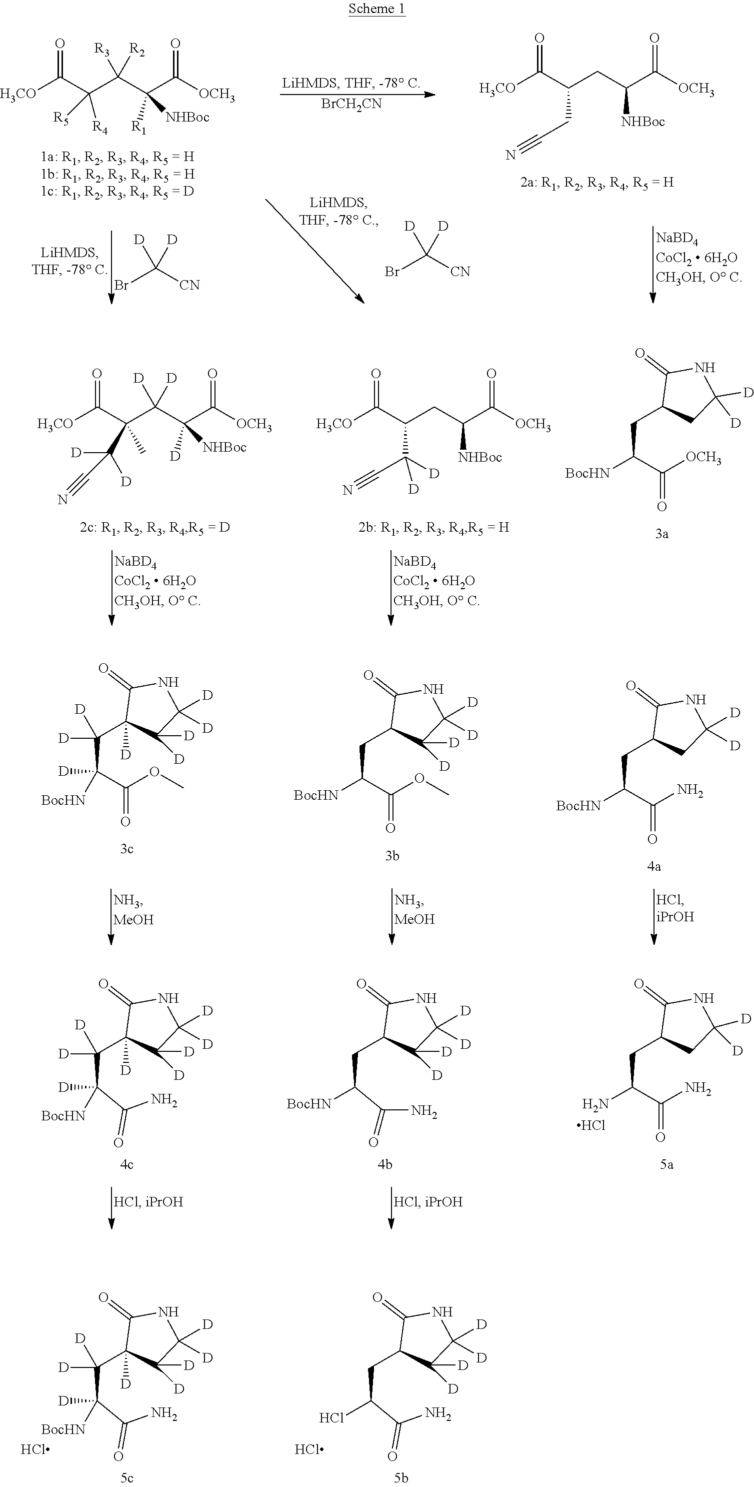

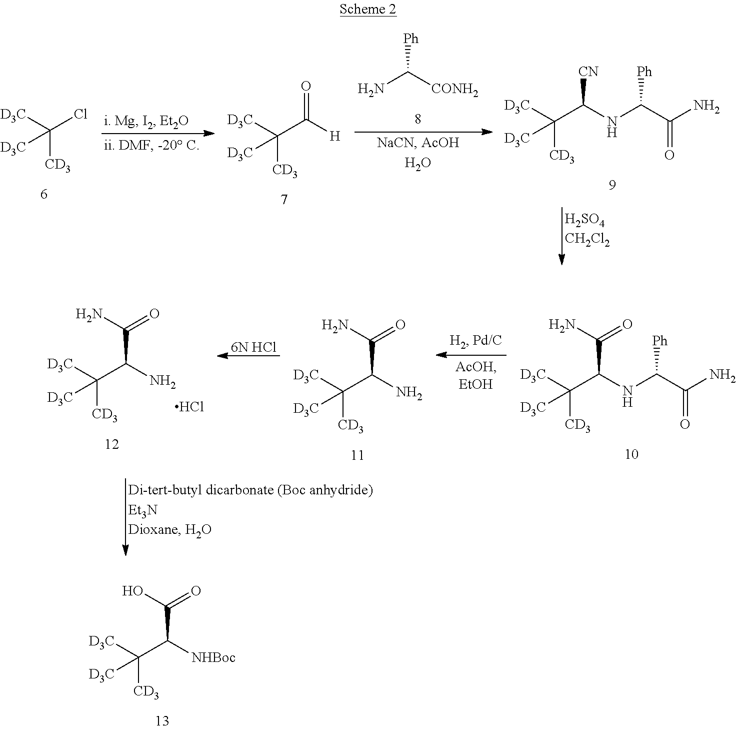

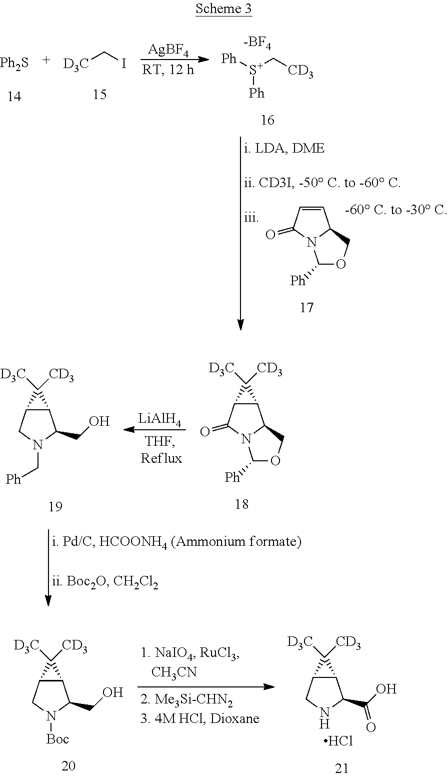

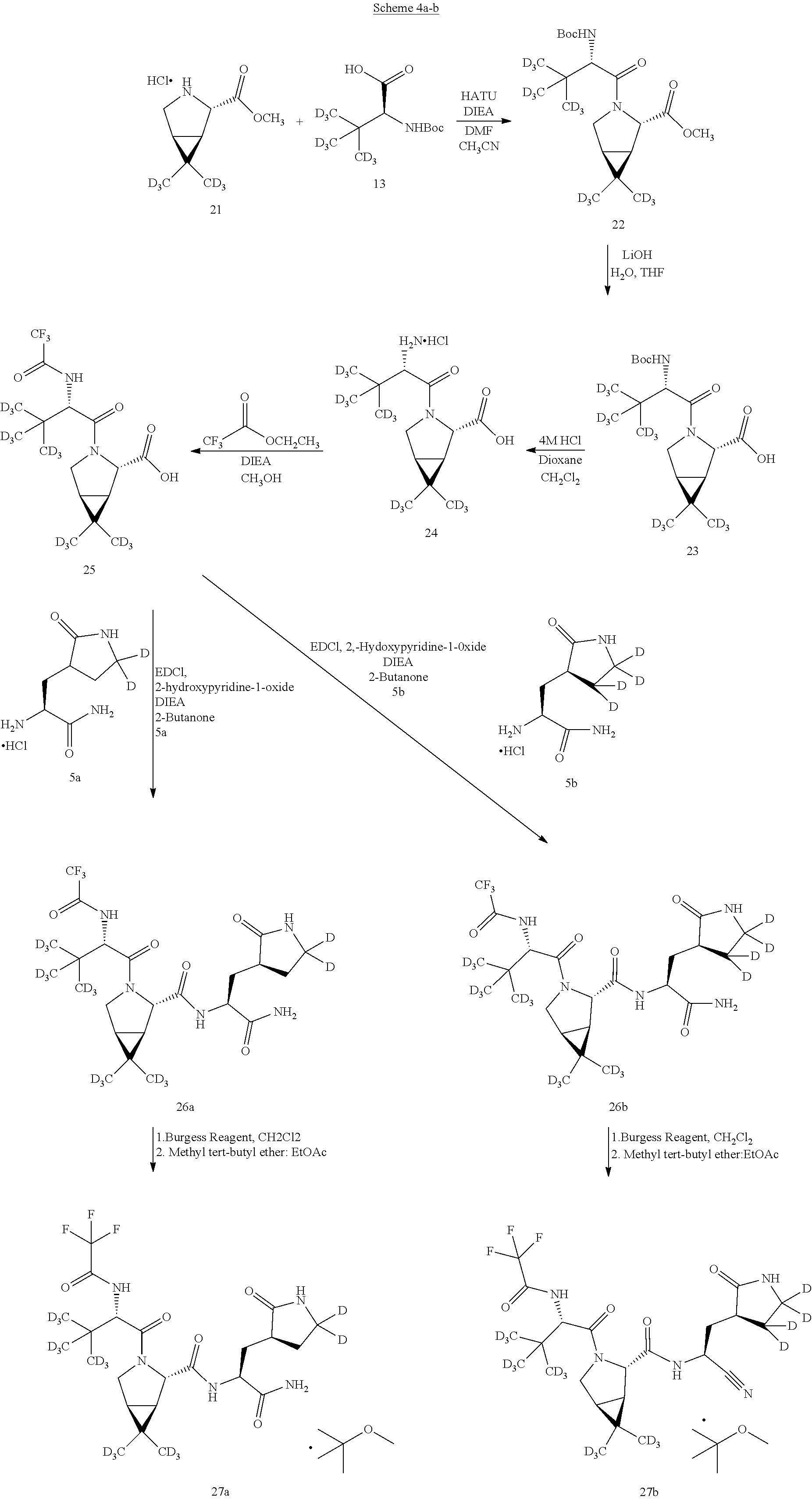

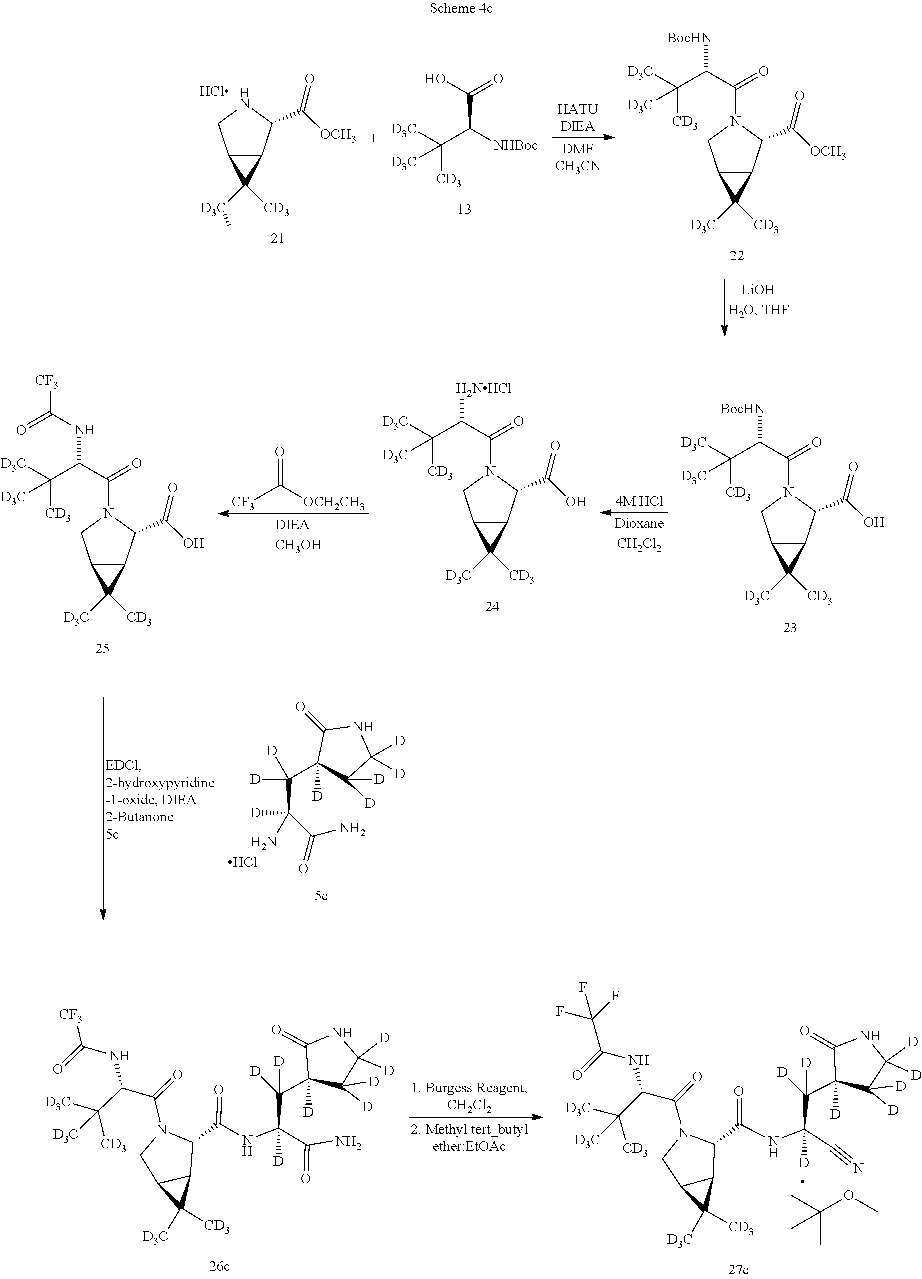

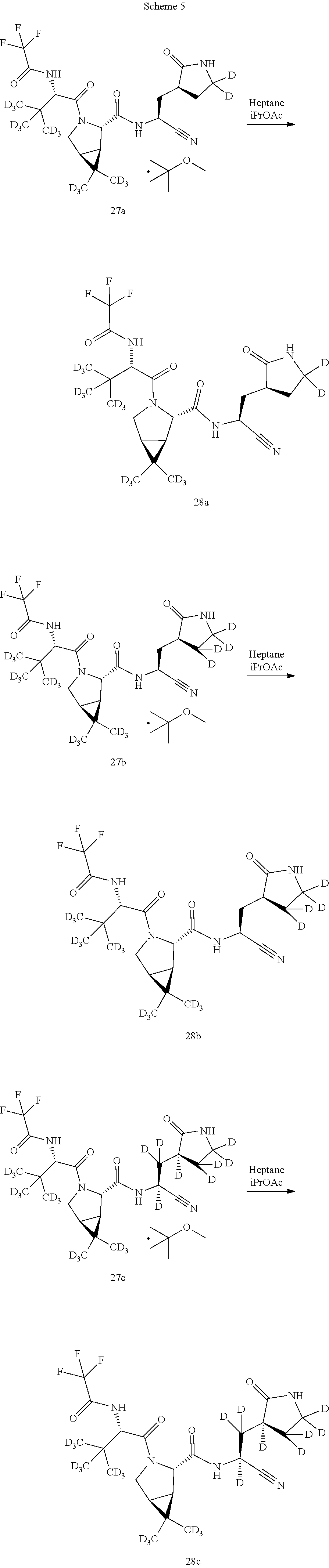

### EXPERIMENTAL

Synthesis of deuterium-enriched compounds of Formula I is described below and illustrated in Schemes 1-5 provided above.

### Synthesis of Deuterated Compounds 5a and 5b (Scheme 1).

### Example 1

### Dimethyl (2S,4R)-2-((tert-butoxycarbonyl)amino)-4-(cyanomethyl)pentanedioate (2a)

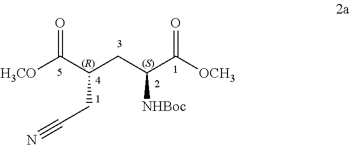

To a solution of N-Boc-L-glutamic acid dimethyl ester 1 (12.0 g, 43.6 mmol) in tetrahydrofuran (THF, 100 mL) at −78° C. is added dropwise a solution of lithium bis(trimethylsilyl)amide (LHMDS) (94 mL, 1 M in THF). The mixture is stirred at −78° C. for 1 h. Subsequently, bromoacetonitrile (3.24 mL, 46.6 mmol) is added dropwise to the mixture at −78° C., and the reaction mixture is stirred at −78° C. for 4 hours. The reaction is quenched with saturated aqueous solution of NH₄Cl (40 mL). The reaction mixture is warmed up to room temperature and extracted with ethyl acetate (50 mL×3). The combined organic extracts are concentrated and purified by flash column chromatography (silica gel, petroleum ether/ethyl acetate=4:1) to yield product 2 (7.6 g, 55%) as a colorless oil. ¹H NMR (CDCl₃): δ 5.11 (d, 1H), 4.38 (s, 1H), 3.77 (s, 3H), 3.75 (s, 3H), 2.92-2.82 (m, 1H), 2.81-2.71 (m, 2H), 2.24-2.08 (m, 2H), 1.44 (s, 9H).

### Example 2

### Methyl (S)-2-((tert-butoxycarbonyl)amino)-3-((S)-2-oxopyrrolidin-3-yl-5,5-d₂)propanoate (3a)

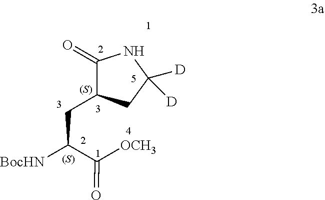

To a stirred solution of 2a (6.0 g, 19.1 mmol) in anhydrous methanol (CH₃OH or MeOH) at 0° C. in a round-bottom flask, is added cobalt (II) chloride hexahydrate (CoCl₂·6H₂O, 2.7 g, 11.36 mmol). Subsequently, sodium borodeuteride (NaBD₄, 4 g, 95.6 mmol mmol) is added portionwise and the reaction mixture is allowed to warm to room temperature and stirred for 12 h. The reaction mixture is quenched with a saturated aqueous solution of NH₄Cl (30 mL). The resulting mixture is concentrated to a residue by evaporating methanol and then extracted with ethyl acetate (50 mL×3). The organic layer is washed with saturated aqueous solution of NH₄Cl (100 mL×3) and brine (100 ml×3). The resulting organic phase is dried over anhydrous MgSO₄, filtered and then concentrated. The resulting organic material is then purified by flash column chromatography (silica gel, petroleum ether/ethyl acetate=2:1) to give the pyrrolidinone compound 3a (2.18 g, 40%) as a white solid. ¹H NMR (CDCl₃): δ 6.65 (s, 1H), 5.55 (s, 1H), 4.3 (d, 1H), 3.71 (s, 3H), 2.47-2.42 (m, 2H), 2.13-2.08 (m, 1H), 1.84-1.81 (m, 2H), 1.41 (s, 9H).

### Example 3

### Synthesis of tert-butyl ((2S)-1-amino-1-oxo-3-((S)-2-oxopyrrolidin-3-yl-5,5-d₂)propan-2-yl)carbamate (4a)

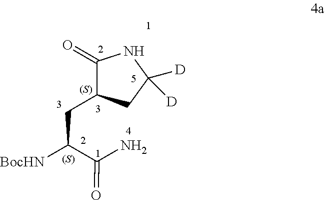

A solution of ammonia (NH₃) in methanol (7 M, 50 ml 36 mmol) was added to Methyl (S)-2-((tert-butoxycarbonyl)amino)-3-((S)-2-oxopyrrolidin-3-yl-5,5-d₂)propanoate (3a) (6.2 g, 22 mmol) and the reaction mixture is stirred at room temperature for 48 h. Concentration of the reaction mixture gives 4a as a yellow solid (5.74 g, 98%). ¹H NMR (CD₃OD) δ 4.10 (dd, 1H), 2.48 (m, 1H), 2.4-2.28 (m, 1H), 2.04 (m 1H), 1.87 (m 1H), 1.74 (m, 1H), 1.45 (s, 9H).

MS m/z 274.3 [M+1].

### Example 4

### Synthesis of (S)-2-amino-3-((S)-2-oxopyrrolidin-3-yl-5,5-d₂)propanamide.HCl Salt (5a)

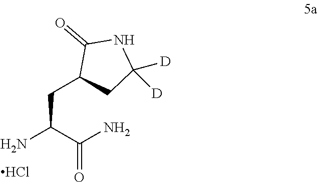 [SMILES: Cl ; [2H]C1([2H])C[C@@H](C[C@H](N)C(N)=O)C(=O)N1]

To a stirred solution of 4a (5.74 g, 18 mol) at 0° C. in isopropanol (iPrOH) (50 mL) is added a solution of HCl in isopropanol (5.5 M; 16 mL, 88 mmol). The reaction mixture is stirred at 50° C. for 4 h, then cooled down to room temperature and stirred at room temperature overnight. The reaction mixture is concentrated on rotary evaporator to yield the aminoamide 5a as HCl salt as a white solid (3.65 g). ¹H NMR (CD₃OD) δ 4.04 (dd, 1H), 2.9-2.68 (m, 1H), 2.43 (m, 1H), 2.21-1.96 (m, 2H), 1.88 (m, 1H). MS m/z 174.2 [M+1].

### Example 5

### Synthesis of(S)-2-amino-3-((S)-2-oxopyrrolidin-3-yl-4,4,5,5-d₄)propanamide. HCl, (5b)

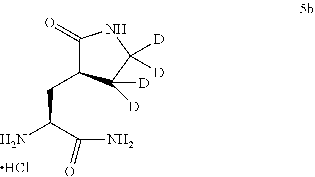 [SMILES: Cl ; [2H]C1([2H])NC(=O)[C@H](C[C@H](N)C(N)=O)C1([2H])[2H]]

Tetradeuterated pyrrolidinone compound 5b is prepared as described in the procedure above for the synthesis of the corresponding dideuterated compound 5a, ((S)-2-amino-3-((S)-2-oxopyrrolidin-3-yl-5,5-d₂)propanamide, and illustrated in Scheme 1, using commercially available starting materials, N-Boc-L-glutamic acid dimethyl ester 1, and 2-bromoacetonitrile-d₂ (Scheme 1). 2-bromoacetonitrile-d₂ can be prepared from alpha deuteration of the 2-bromoaceonitrile.

To a stirred solution of 4b (5.74 g, 18 mol) at 0° C. in isopropanol (iPrOH) (50 mL) is added a solution of HCl in isopropanol (5.5 M; 16 mL, 88 mmol). The reaction mixture is stirred at 50° C. for 4 h, then cooled down to room temperature and stirred at room temperature overnight. The mixture is concentrated on rotary evaporator to yield the aminoamide 5b as HCl salt as a white solid (3.6 g). ¹H NMR (CD₃OD) δ 4.04 (dd, 1H), 2.9-2.68 (m, 1H), 2.43 (m, 1H), 1.88 (m, 1H). MS m/z 176.2 [M+1].

### Example 6

### Synthesis of (S)-2-amino-3-((S)-2-oxopyrrolidin-3-yl-3-d₁,4,4-d₂,5,5-d₂)propanamide-2-d₁,3,3-d₂. HCl, (5c)

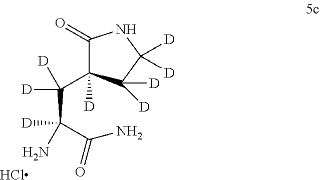

Octadeuterated (d₈) pyrrolidinone compound 5c is synthesized as described in the procedure above for the synthesis of the corresponding deuterated compounds 5a and 5b, and illustrated in Scheme 1, from readily available starting materials, deuterated N-Boc-L-glutamic acid dimethyl ester-d₅ 1, and 2-bromoacetonitrile-d₂ (BrCD₂CN or BrCH₂CN-d₂, shown in Scheme 1). 2-bromoacetonitrile-d₂ is prepared from alpha deuteration of the readily commercially available 2-bromoaceonitrile (BrCH₂CN).

To a stirred solution of 4c (5.7 g, 18 mol) at 0° C. in isopropanol (iPrOH) (50 mL) is added a solution of HCl in isopropanol (5.5 M; 16 mL, 88 mmol). The reaction mixture is stirred at 50° C. for 4 h, then cooled down to room temperature and stirred at room temperature overnight. The mixture is concentrated on rotary evaporator to yield the aminoamide 5c as HCl salt as a white solid (3.6 g). MS m/z 180.2 [M+1].

### Synthesis of Compound 13 (Scheme 2)

### Example 7

### 2,2-Dimethyl-d₆-propanal-3,3,3-d₃ (7)

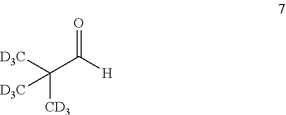 [SMILES: [2H]C([2H])([2H])C(C=O)(C([2H])([2H])[2H])C([2H])([2H])[2H]]

In a 300 mL 3-necked round bottom flask fitted with a reflux condenser, dropping funnel (and a mechanical stirrer for large scale, if a magnetic stir bar is not sufficient for stirring of the reaction mixture) is placed a few small crystals of iodine and then magnesium turnings (2.47 g, 0.103 mol). The bottom of the flask was heated cautiously with a heat gun until the iodine began to vaporize and which is then allowed to cool down. A solution of commercially available deuterated t-butyl-d₉ chloride, 6 (10 g, 0.103 mol, 99 atom % D) in anhydrous diethyl ether (Et₂O or ether) is placed in the dropping funnel. A small amount of the solution of t-butyl chloride-d₉ in ether (1 ml) is added directly to the dry magnesium in the reaction flask. Anhydrous ether (100 mL) and a few small crystals of iodine are added, and the resulting mixture is heated for a few minutes to initiate the Grignard reaction. The rest of the solution of t-butyl chloride-d₉ in ether is added with stirring slowly at a rate of approximately one drop per second. The mixture is allowed to reflux on its own during the addition without any external cooling. The reaction mixture is then heated at reflux for several hours until almost all of the magnesium is consumed. The mixture is then cooled to −20° C., and a solution of anhydrous DMF (7.3 g, 0.1 mol) in ether (100 mL) is added at such a rate that the temperature of the reaction not exceed −15° C. A second solution of anhydrous DMF (7.3. g, 0.1 mol) is then quickly added at −10° C. After 5 min, hydroquinone (500 mg) is added, and then stirring is stopped, the cooling bath is removed, and the mixture is left standing overnight at room temperature under nitrogen. The mixture is then cooled to 5° C., and aqueous 4 N HCl solution (60 mL) is added slowly and cautiously in portions to quench the reaction. The mixture is diluted with water (40 mL), and the organic and aqueous layers are separated. The aqueous layer is extracted with ether (3×25 mL), and the combined organic layers are dried with anhydrous MgSO₄ and then filtered. The filtrate is concentrated cautiously by fractional distillation under an atmosphere of nitrogen to remove most of the solvent ether to collect the deuterated triacetaldehyde-d₉ (7) at 65-75° C. (3.95 g) as a colorless oil. Deuterated aldehyde 7 is stored under nitrogen in the freezer and used in the next step.

### Example 8

### (R)-2-(((S)-1-cyano-2,2-(dimethyl-d₆)propyl-3,3,3-d₃)amino)-2-phenylacetamide (9)

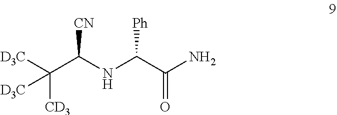

To a stirred suspension of commercially available (R)-phenylglycine amide, 8, (6.7 g, 40 mmol) in water (40 mL) is added compound 7 (3.95 g, 41.5 mmol) at room temperature. Simultaneously, 30% aqueous NaCN solution (6.88 g, 42 mmol) and glacial acetic acid (2.54 g, 42.3 mmol) are added slowly. The resulting mixture is stirred for 2 h, followed by stirring at 70° C. for 20 h. After cooling to room temperature the product is isolated by filtration. The solid is washed with water (50 mL) and dried in vacuo to afford deuterated aminonitrile 9 (9.0 g,) as a tan solid with [α]_{D}=−298° (c=1.0, CHCl₃). MS (ESI) 255.3 (M+1).

### Example 9

### (S)-2-(((2R)-2-Amino-2-oxo-1-phenylethyl)amino)-3,3-dimethylbutanamide-d₉ (10)

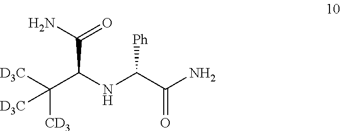 [SMILES: [2H]C([2H])([2H])C([C@H](N[C@@H](C(N)=O)c1ccccc1)C(N)=O)(C([2H])([2H])[2H])C([2H])([2H])[2H]]

To a solution of compound 9 (6.42 g, 25.2 mmol) in dichloromethane (50 ml) at 0° C. is added conc. sulfuric acid (35 mL) at 15-20° C. through an addition funnel and the mixture stirred at room temperature for 1 h. The mixture is then poured on to ice and carefully treated with NH₄OH solution to adjust to pH 9 of the mixture. The mixture is extracted with dichloromethane, and the combined organic layers are washed with water, dried over anhydrous Na₂SO₄, filtered, and concentrated in vacuo to yield bisamide 10 (5.5.g) as a yellow foam with [α]_{D}=˜140′ (c=1.0, CHCl₃). MS (ESI) 273.3 (M+1).

### Example 10

### (S)-2-amino-(3,3)-dimethylbutanamide-d₉ (11)

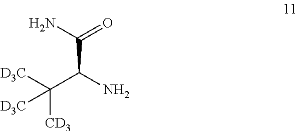 [SMILES: [2H]C([2H])([2H])C([C@H](N)C(N)=O)(C([2H])([2H])[2H])C([2H])([2H])[2H]]

A mixture of compound 10 (7.7 g, 28.3 mmol), 10% Pd/C (˜50% water, 2 g), and acetic acid (5 ml) in ethanol (120 mL) is shaken under hydrogen gas at 30 psi at room temperature for several days by using hydrogen gas apparatus. After completion of the reaction, the mixture is filtered through Celite® and washed with ethyl acetate. The filtrate is then concentrated in vacuo, the resulting residue is diluted with water (100 mL) and basified with 1 N NaOH solution to pH 9. The mixture is extracted with dichloromethane, and the aqueous layer is concentrated in vacuo to half its initial volume, saturated with solid NaCl, and extracted with THF. The combined organic extracts are dried with Na₂SO₄, filtered, and concentrated in vacuo. The resulting residue is diluted with toluene and subsequently evaporated to remove the remaining water, followed by trituration with dichloromethane to produce the aminoamide 11 (3.8 g) as a white solid, which is used in the next step without further purification.

### Example 11

### (S)-2-Amino-3,3-dimethylbutanoic acid-d₉ hydrochloride (12)

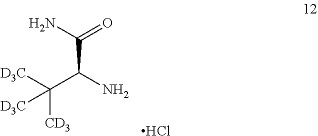 [SMILES: [2H]C([2H])([2H])C([C@H](N)C(N)=O)(C([2H])([2H])[2H])C([2H])([2H])[2H] ; Cl]

A mixture of compound 11 (3.1 g, 22.3 mmol) in 6 N aqueous HCl solution (150 ml) is refluxed for 24 h. The mixture is concentrated in vacuo to produce the crude product. The solid is redissolved in water (50 mL) and washed with ethyl acetate (2×20 mL) to remove impurities from previous steps. The aqueous layer is then concentrated in vacuo, diluted with toluene, evaporated, and dried in vacuo at 50° C. to give the HCl salt of the desired compound (S)-2-amino-3,3-dimethylbutanoic acid-d₉ hydrochloride 12 (3.4 g) as a white solid, which is used in the next step without further purification.

### Example 12

(S)-2-((tert-butoxycarbonyl)amino)-3,3-(dimethyl-d₆)butanoic-4,4,4-d₃ acid (13)

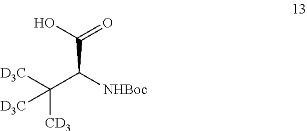 [SMILES: [2H]C([2H])([2H])C([C@H](NC(=O)OC(C)(C)C)C(=O)O)(C([2H])([2H])[2H])C([2H])([2H])[2H]]

To a solution of compound 12 (1.00 g, 5.66 mmol) in a mixture of dioxane (10 mL) and water (10 mL) is added triethylamine (3.16 ml, 22.6 mmol) followed by di-tert-butyl dicarbonate (1.48 g, 6.79 mmol). The resulting mixture is stirred at room temperature for 6 h, and then washed with heptane (2×20 mL). The aqueous phase is cooled, acidified by adding 1 N HCl to pH 2, and t h e n extracted with ethyl acetate (3×50 mL). The combined extracts are dried over Na₂SO₄, filtered, and concentrated in vacuo to yield Boc-tert-leucine-d₉ 13 (1.10 g) as yellow oil. MS (ESI) 239.2 (M−1).

### Synthesis of Compound 21 (Scheme 3)

### Example 13

### Diphenyl(2,2,2-trideuteroethyl-d₃)sulfonium tetrafluoroborate (16)

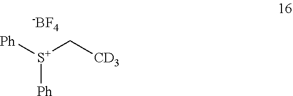 [SMILES: "-BF4" ; [2H]C([2H])([2H])C[S+](c1ccccc1)c1ccccc1]

A solution of diphenyl sulfide, 14, (2.32 ml, 13.8 mmol) and CH₂Cl₂ (15 mL) is added to a flask containing commercially available 2,2,2-iodoethane-d₃, 15 (10 g, 62.9 mmol, 99 atom % D). The mixture is cooled to 0° C. and stirred vigorously. To the cooled mixture is added silver tetrafluoroborate (2.45 g, 12.6 mmol) in one portion, and the mixture is warmed to ambient temperature over 24 h. The mixture is then diluted with CH₂Cl₂ (100 mL) and passed through a pad of Celite® to remove the precipitated silver iodide. The filtrate is concentrated in vacuo, and the crude oil triturated with diethyl ether (Et₂O) to afford a white solid. Recrystallization of the crude material from ethanol (EtOH) yields 16 as a white powder (3.3 g, 86% yield). MS (ESI) 218.2 [M⁺].

### Example 14

### (3R,5αS,6αS,6βS)-3-Phenyl-6,6-bis(trideuteromethyl-d₃)tetrahydro-1H-cyclopropa[3,4]pyrrolo[1,2-c]oxazol-5(3H)-one (18)

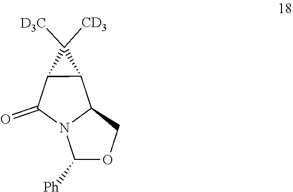 [SMILES: [2H]C([2H])([2H])C1(C([2H])([2H])[2H])[C@@H]2[C@H]3CO[C@H](c4ccccc4)N3C(=O)[C@@H]21]

To a solution of freshly prepared LDA (0.94 M) in THF (13.0 mL, 12.4 mmol) at −70° C. is added a solution of 16 (3.64 g, 11.9 mmol) in DME (24 ml). The solution is stirred at −60° C. for half hour, followed by the addition of iodomethane-d₃ (0.740 ml, 11.9 mmol, Sigma-Aldrich, 99.5 atom % D). The mixture is then warmed slowly to −50° C. and stirred at −50° C. for a period of 2 h. The reaction mixture is then recooled to −70° C., and a solution of LDA (0.94 M) in THF (13.8 mL, 13.1 mmol) is added and the mixture is stirred at −70° C. for a period of 1 h. To the stirred solution at −70° C. is then added a solution of commercially available (3R,7αS)-3-phenyl-1,7a-dihydropyrrolo[1,2-c]oxazol-5(3H)-one (17; 0.96 g, 4.77 mmol) in DME (9 mL). The reaction mixture is stirred for 1 h at −70° C., followed by warming to −30° C. and stirred for 2 hours. The mixture is then diluted with saturated aqueous solution of NaHCO₃ (30 mlL) and warmed to room temperature. The mixture is extracted with diethyl ether (3×25 mL) and the extracts are dried over Na₂SO₄, filtered, and concentrated in vacuo. Purification of the crude product by column chromatography (Silica gel, EtOAc/heptane) gives 18 as a white solid (0.98 g).MS (ESI) 250.1 (M+1).

### Example 15

### ((1R,2S,5S)-3-Benzyl-6,6-(dimethyl-d₆)-3-azabicyclo[3.1.0]hexan-2-yl)methanol (19)

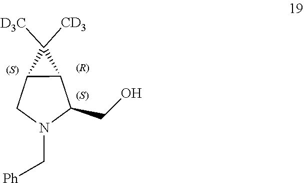

To a solution of lactam-d₆ 18 (376 mg, 1.51 mmol) in THF (3 mL) at 0° C. is added LiAlH₄ (2 M in THF, 1.51 mL, 3.02 mmol). The reaction is refluxed for 3 h, then cooled to 0° C., and quenched by dropwise addition of 10% aqueous solution of KHSO₄. The resulting slurry is diluted with ethyl acetate and filtered (washing the filter cake with ethyl acetate (2×10 mL). The resulting solution is diluted with water (20 ml) and extracted with ethyl acetate (3×30 ml). The combined organic phase is washed with brine, dried with MgSO₄, and concentrated in vacuo to yield the hexadeuterated bicyclic prolinol-d₆ 19 (0.35 g). (ESI) 238.3 (M+1).

### Example 16

### tert-butyl (1R,2S,5S)-2-(hydroxymethyl)-6,6-(dimethyl-d₆)-3-azabicyclo[3.1.0]hexane-3-carboxylate (20)

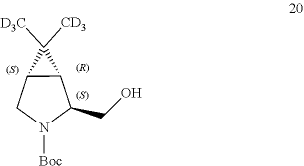

To a solution of hexadeuterated bicyclic prolinol-d₆ 19 (350 mg,1.48 mmol) in 15 mL of methanol is added ammonium formate (571 mg, 9.06 mmol) followed by 10% palladium on carbon (70 mg, 20 wt. %). The reaction mixture is then heated at reflux for 2 h, taking precautions to limit ammonium formate sublimation inside the condenser. The reaction mixture is cooled to room temperature and filtered through Celite®. The Celite® pad is washed with methanol (2×10 mL) and then with dichloromethane (2×20 mL). The resulting filtrate is then concentrated in vacuo to give the desired de-benzylated bicyclic prolinol-d6 (1.48 mmol), which then is dissolved in dichloromethane (5 mL), and triethylamine (273 μL, 1.96 mmol) is added followed by di-tert-butyl dicarbonate (428 mg, 1.96 mmol). The reaction is stirred at room temperature for 15 h, diluted with 1 N HCl (15 mL), and then extracted with dichloromethane (3×30 mL). The combined organic layers are washed with brine, dried over Na₂SO₄, filtered, and concentrated in vacuo. The resulting residue is purified by column chromatography (Silica gel, 0-20% EtOAc/heptane) to yield N-boc protected gem-dideuteromethyl-bicyclic prolinol-d₆ 20 (311 mg,). (ESI) 270.2 [M+Na]⁺.

### Example 17

### Methyl (1R,2S,5S)-6,6-(dmethyl-d₆)-3-azabicyclo[3.1.0]hexane-2-carboxylate hydrochloride (21)

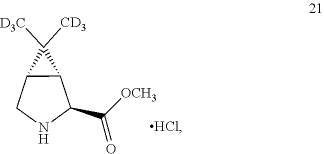 [SMILES: [2H]C([2H])([2H])C1(C([2H])([2H])[2H])[C@@H]2[C@@H](C(=O)OC)NC[C@@H]21]

To a solution of N-boc protected gem-dideuteromethyl(d₆)-bicyclic prolinol20 (311 mg, 1.26 mmol) in ethylacetate (10 mL) and acetonitrile (10 mL) is added a solution of ruthenium trichloride monohydrate (5.50 mg, 0.0252 mmol) and sodium periodate (2.16 g, 10.0 mmol) in water (15 mL). After stirring at room temperature for 1 h, the reaction is filtered through Celite® and filter pad is washed with ethyl acetate (3×5 mL). The resulting filtrate is concentrated to a residue, which is diluted with 1 M HCl (10 mL) and extracted with ethyl acetate (3×20 mL). The organic extracts are combined, washed with 1 M HCl, dried over Na₂SO₄, filtered, and concentrated to give a light yellowish solid. The crude product (313 mg, 1.20 mmol) is dissolved in a mixture of benzene (5.0 mL) and methanol (0.50 ml), and a 2 M solution of trimethylsilyl diazomethane (TMSCHN₂) in hexanes (780 μL, 1.56 mmol) is added dropwise. The yellow solution is stirred at room temperature for 15 h and is subsequently quenched by dropwise addition of acetic acid until effervescence ceases. The reaction is then concentrated in vacuo with several repeated heptane dilutions/concentrations to remove excess acetic acid. The resulting residue is then purified by column chromatography (Silica gel, 0-30% ethyl acetate/heptane) to yield pure bicyclic proline methyl ester-d₆ (154 mg). To this methyl ester-d₆ is added a 4 M solution of HCl (or 4N HCl) in dioxane (5.0 mL), and the resulting solution is stirred at room temperature for 2 h. The reaction is then concentrated in vacuo to produce pure hexadeuterated bicyclic proline methyl ester 21 (Scheme 3) as its hydrochloride salt (128 mg) as a colorless solid. MS (ESI) m/z 176.2 (M+1).

### Synthesis of Compounds 27a and 27b (Scheme 4)

### Example 18

### Methyl (1R,2S,5S)-3-((S)-2-((tert-butoxycarbonyl)amino)-3,3-bis(methyl-d₃)butanoyl-4,4,4-d₃)-6,6-bis(methyl-d₃)-3-azabicyclo[3.1.0]hexane-2-carboxylate (22)

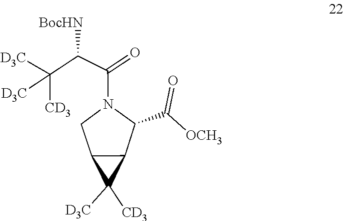 [SMILES: [2H]C([2H])([2H])C1(C([2H])([2H])[2H])[C@@H]2[C@@H](C(=O)OC)N(C(=O)[C@@H](NC(=O)OC(C)(C)C)C(C([2H])([2H])[2H])(C([2H])([2H])[2H])C([2H])([2H])[2H])C[C@@H]21]

To a solution of methyl (1R,2S,5S)-6,6-dimethyl-3-azabicyclo[3.1.0]hexane-2-carboxylate, HCl salt 21 (5 g, 24.3 mmol) and N-Boc-tert-leucine-d₉ (13) (or may also be known as N-(tert-butoxycarbonyl)-3-methyl-d₃-L-valine-d₆) (6.18 g, 26.7 mmol) in a mixture of N,N-dimethylformamide (DMF, 97 mL) and acetonitrile (875 mL) at 0° C. is added HATU (10.2 g, 26.7 mmol, Sigma-Aldrich), followed by drop-wise addition of N,N-diisopropylethylamine (12.7 mL, 72.9 mmol). The reaction mixture is then allowed to warm to 25° C. and is stirred for 16 h, and then concentrated. The resulting residue is treated with ethyl acetate (20 mL), washed with water (20 ml). The separated aqueous phase is extracted with ethyl acetate (2×200 mL). The combined organic phases are washed with water (20 mL) and HCl (1 M; 10 mL), saturated aqueous sodium chloride solution (10 mL), dried over sodium sulfate, filtered, and concentrated. The residue is purified using silica gel chromatography (30% to 50% gradient ethyl acetate in heptane) to yield 22 as colorless oil (8.9 g, 95%). ¹H NMR (DMSO-d₆) δ 6.73 (d, 1H), 4.21 (s, 1H), 4.05 (d, 1H), 3.93 (d, 1H), 3.79 (dd, 1H), 3.65 (s, 3H), 1.55-1.49 (m, 1H), 1.41 (d, 1H), 1.35 (s, 9H). MS m/z 398.3 [M+1].

### Example 19

### (1R,2S,5S)-3-((S)-2-((tert-butoxycarbonyl)amino)-3,3-bis(methyl-d₃)butanoyl-4,4,4-d₃)-6,6-bis(methyl-d₃)-3-azabicyclo[3.1.0]hexane-2-carboxylic acid (23)

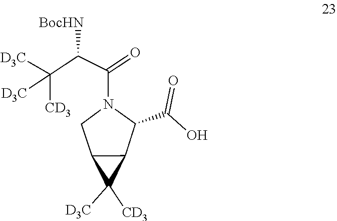 [SMILES: [2H]C([2H])([2H])C1(C([2H])([2H])[2H])[C@@H]2[C@@H](C(=O)O)N(C(=O)[C@@H](NC(=O)OC(C)(C)C)C(C([2H])([2H])[2H])(C([2H])([2H])[2H])C([2H])([2H])[2H])C[C@@H]21]

To a solution of 22 (8.8 g, 23 mmol) in tetrahydrofuran (25 mL) is added lithium hydroxide (1.66 g, 6.92 mmol) and water (5 mL and reaction mixture is stirred at room temperature for 2 h. Most of tetrahydrofuran is removed in vacuo and the residue is then acidified to pH 2 by addition of 1 M HCl. The resulting mixture is extracted with ethyl acetate (2×20 mL), and the combined organic layers are washed with saturated aqueous sodium chloride solution (50 mL, dried over sodium sulfate, filtered, and concentrated to provide 23 as a white solid (8.5 g, 100%). ¹H NMR (DMSO-d₆) δ 12.5 (s, 1H), 6.67 (d, 1H), 4.13 (s, 1H), 4.04 (s, 1H), 3.91 (d, 1H), 3.77 (dd, 1H), 1.54-1.46 (m, 1H), 1.40 (s, 1H), 1.35 (s, 9H). MS m/z 384.3 [M+1].

### Example 20

### (1R,2S,5S)-3-((S)-2-amino-3,3-bis(methyl-d₃)butanoyl-4,4,4-d₃)-6,6-bis(methyl-d₃)-3-azabicyclo[3.1.0]hexane-2-carboxylic acid.HCl (24)

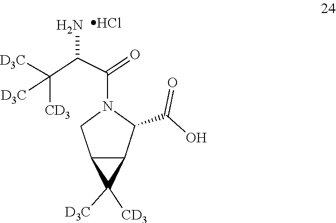 [SMILES: Cl ; [2H]C([2H])([2H])C1(C([2H])([2H])[2H])[C@@H]2[C@@H](C(=O)O)N(C(=O)[C@@H](N)C(C([2H])([2H])[2H])(C([2H])([2H])[2H])C([2H])([2H])[2H])C[C@@H]21]

A solution of HCl in 1,4-dioxane (27.5 mL of 4 N HCl) is added to a solution of 24 (8.1 g, 21.9 mmol) in dichloromethane (22 mL), and the reaction mixture is stirred at room temperature for 16 h. organic solvents are removed to yield 24 as its HCl salt as a white solid (6.65 g, 99%). ¹H HNMR (DMSO-d₆) δ 12.81 (m, 1H), 8.18 (br s, 2H), 4.18 (s, 1H), 3.86-3.74 (m, 2H), 3.71 (d, 1H), 1.60-1.51 (m, 1H), 1.46 (d, 1H). MS m/z 284.3 [M+1].

### Example 21

### (1R,2S,5S)-3-((S)-3,3-bis(methyl-d₃)-2-(2,2,2-trifluoroacetamido)butanoyl-4,4,4-d₃)-6,6-bis(methyl-d₃)-3-azabicyclo[3.1.0]hexane-2-carboxylic acid (25)

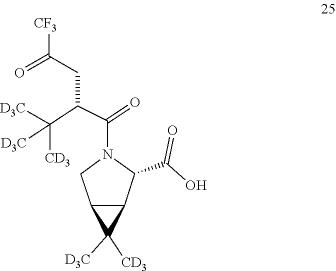 [SMILES: [2H]C([2H])([2H])C1(C([2H])([2H])[2H])[C@@H]2[C@@H](C(=O)O)N(C(=O)[C@@H](CC(=O)C(F)(F)F)C(C([2H])([2H])[2H])(C([2H])([2H])[2H])C([2H])([2H])[2H])C[C@@H]21]

To a 0° C. solution of the HCl salt of 24 (5.5 g, 28 mmol) in methanol (20 mL) is added triethylamine (15 mL), followed by ethyl trifluoroacetate (6.4 g, 45 mmol), and the reaction mixture is allowed to warm to 50° C., and is stirred for 16 h. The reaction mixture is concentrated in vacuo at 50° C., and the residue is diluted with water (25 ml) and acidified to a pH of 3 to 4 by addition of 1 M HCl (25 mL) followed by conc. HCl. After extraction of the aqueous layer with ethyl acetate (3×25 mL), the combined organic layers are washed with saturated aqueous sodium chloride solution (30 mL), dried over sodium sulfate, filtered, and concentrated to afford 25 as a white solid (7 g). ¹H NMR (DMSO-d₆) δ 12.99-12.47 (m, 1H), 9.43 (d, 1H), 4.44 (d, 1H), 4.15 (s, 1H), 3.85 (dd, 1H), 3.72 (d, 1H), 1.53 (dd, 1H), 1.43 (d, 1H). MS m/z 380.1 [M+1].

### Example 22

### (1R,2S,5S)-N-((S)-1-amino-1-oxo-3-((S)-2-oxopyrrolidin-3-yl-5,5-d₂)propan-2-yl)-3-((S)-3,3-bis(methyl-d₃)-2-(2,2,2-trifluoroacetamido)butanoyl-4,4,4-d₃)-6,6-bis(methyl-d₃)-3-azabicyclo[3.1.0]hexane-2-carboxamide (26a)

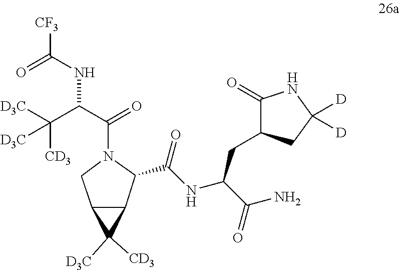 [SMILES: [2H]C1([2H])C[C@@H](C[C@H](NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(N)=O)C(=O)N1]

To a stirring solution of 25 (6 g, 13.5 mmol) and 5a (3.35 g, 15 mol) in 2-butanone (50 ml) is added 2-Hydroxypyridine 1-oxide (0.37 g, 3.4 mmol) and the mixture is cooled to 0° C. N,N-Diisopropylethylamine (DIEA, 7.5 mL) is then added, followed by the addition of EDCI (3 g). The reaction mixture is stirred at room temperature for 16 h, and then diluted with ethyl acetate/tert-butyl methyl ether (1:1, 50 mL) and washed with a mixture of water (20 mL) and saturated aqueous sodium chloride solution (10 mL). The separated organic layer is washed with saturated aqueous sodium chloride solution (30 ml), followed by a mixture of HCl (20 mL of 1M) and saturated aqueous sodium chloride solution (10 mL), then saturated aqueous sodium chloride solution (30 mL), dried over magnesium sulfate, filtered, and concentrated to give 26a as a white solid (7 g). ¹H NMR (DMSO-d₆) δ 9.4 (d, 1H), 8.29 (d, 1H), 7.5 (s, 1H), 7.3 (br s, 1H), 7 (br s, 1H), 4.4 (d, 1H), 4.35-4.25 (m, 2H), 3.91-3.84 (m, 1H), 3.67 (d, 1H), 2.45-2.34 (m, 1H), 2.14 (dt,1H), 1.97-1.86 (m, 1H), 1.70-1.57 (m, 1H), 1.54-1.4 (m, 2H), 1.38 (d, 1H). MS m/z 535.4 [M+1].

### Example 22

### (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl)-6,6-bis(methyl-d₃)-3-azabicyclo[3.1.0]hexane-2-carboxamide (t-butyl methyl ether solvate, MTBE) (27a)

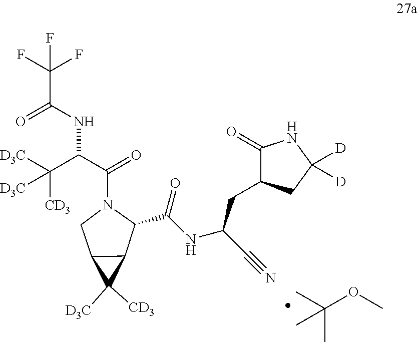

To a solution of 26a (6.1 g, 11.1 mmol) in dichloromethane (55 mL) is added Methyl N-(triethylammoniosulfonyl)carbamate, inner salt (Burgess reagent, 6.93 g, 27.6 mmol) and the reaction mixture stirred at room temperature for 1 h. A mixture of saturated aqueous sodium bicarbonate solution (20 mL) and saturated aqueous sodium chloride solution (10 mL) is added to the reaction mixture. The organic phase is separated and concentrated. The resulting residue is dissolved in 50% ethyl acetate/tert-butyl methyl ether (60 mL), washed with a mixture of saturated aqueous sodium bicarbonate solution (20 mL) and saturated aqueous sodium chloride solution (10 mL) twice, saturated aqueous sodium chloride solution (20 mL), a mixture of HCl (20 mL of 1N HCl) and saturated aqueous sodium chloride solution (10 mL) twice. The organic layer is then dried over magnesium sulfate, filtered, and concentrated. The residue is treated with a mixture of ethyl acetate and tert-butyl methyl ether (1:10, 40 mL) and heated to 50° C.; after stirring for 1 hour at 50° C., it is cooled to 25° C. and stirred overnight. The solid is collected by filtration, dissolved in dichloromethane (10 ml) and filtered through silica gel (20 g); the silica gel is then washed with ethyl acetate (100 mL), 10% methanol in ethyl acetate (200 mL). The combined eluates are concentrated and a mixture of ethyl acetate and tert-butyl methyl ether (5:95, 55 ml) is added. This mixture is heated to 50° C. for 1 h, cooled to 25° C., and stirred overnight, which is then filtered to yield 27a, (MTBE solvate), as a white solid (10.4 g, 75%). ¹H NMR (DMSO-d₆) δ 9.43 (d, 1H), 9.03 (d, 1H), 7.68 (s, 1H), 4.97 (ddd, 1H), 4.4 (d, 1H), 4.15 (s, 1H), 3.91 (dd, 1H), 3.69 (d, 1H), 3.07 (s, 3H, MTBE), 2.4 (tdd, 1H), 2.14 (ddd, 1H), 2.11-2.03 (m, 1H), 1.76-1.65 (m, 2H), 1.57 (dd, 1H), 1.32 (d, 1H), 1.10 (s, 9H, MTBE).

The deuterated compounds 26b and 27b are prepared from compounds 25 and 5b using the same procedures as described above for the synthesis of the corresponding deuterated compounds 26a and 27a from 25 and 5a (Scheme 4a-b).

### Example 23

### (1R,2S,5S)-N-((S)-1-amino-1-oxo-3-((S)-2-oxopyrrolidin-3-yl-4,4,5,5-d₄)propan-2-yl)-3-((S)-3,3-bis(methyl-d₃)-2-(2,2,2-trifluoroacetamido)butanoyl-4,4,4-d₃)-6,6-bis(methyl-d₃)-3-azabicyclo[3.1.0]hexane-2-carboxamide (26b)

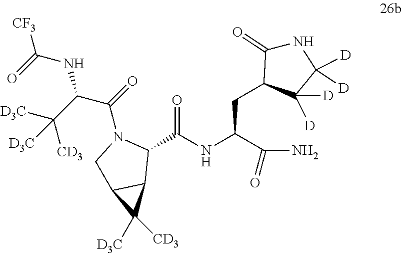 [SMILES: [2H]C([2H])([2H])C1(C([2H])([2H])[2H])[C@@H]2[C@@H](C(=O)N[C@@H](C[C@H]3C(=O)NC([2H])([2H])C3([2H])[2H])C(N)=O)N(C(=O)[C@@H](NC(=O)C(F)(F)F)C(C([2H])([2H])[2H])(C([2H])([2H])[2H])C([2H])([2H])[2H])C[C@@H]21]

To a stirring solution of 25 (6 g, 13.5 mmol) and the HCl salt of 5b (3.35 g, 15 mol) in 2-butanone (50 ml) is added 2-Hydroxypyridine 1-oxide (0.37 g, 3.4 mmol) and the mixture is cooled to 0° C. N,N-Diisopropylethylamine (DIEA, 7.5 mL) is then added, followed by the addition of EDCI (3 g). The reaction mixture is stirred at room temperature for 16 h, and then diluted with ethyl acetate/tert-butyl methyl ether (1:1, 50 mL) and washed with a mixture of water (20 mL) and saturated aqueous sodium chloride solution (10 mL). The separated organic layer is washed with saturated aqueous sodium chloride solution (30 ml), followed by a mixture of HCl (20 mL of 1M) and saturated aqueous sodium chloride solution (10 mL), then saturated aqueous sodium chloride solution (30 ml), dried over magnesium sulfate, filtered, and organic phase is then concentrated to give 26b as a white solid (7 g,). ¹H NMR (DMSO-d₆) δ 9.4 (d, 1H), 8.29 (d, 1H), 7.5 (s, 1H), 7.3 (br s, 1H), 7 (br s, 1H), 4.4 (d, 1H), 4.35-4.25 (m, 2H), 3.91-3.84 (m, 1H), 3.67 (d, 1H), 2.45-2.34 (m, 1H), 1.70-1.57 (m, 1H), 1.54-1.4 (m, 2H), 1.38 (d, 1H). MS m/z 537.4 [M+1].

### Example 24

### (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-carboxamide (t-butyl methyl ether solvate, MTBE) (27b)

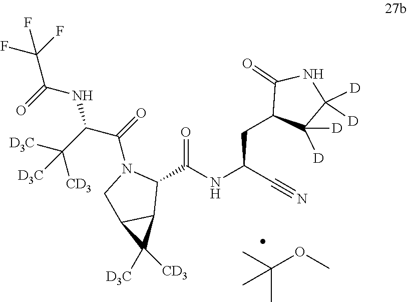

To a solution of 26b (6.1 g, 111. mmol) in dichloromethane (55 ml) is added Methyl N-(triethylammoniosulfonyl)carbamate, inner salt (Burgess reagent, 6.93 g, 27.6 mmol) and the reaction mixture stirred at room temperature for 1 h. A mixture of saturated aqueous sodium bicarbonate solution (20 mL) and saturated aqueous sodium chloride solution (10 mL) is added to the reaction mixture. The organic phase is separated and concentrated. The resulting residue is dissolved in in 50% ethyl acetate/tert-butyl methyl ether (60 mL), washed with a mixture of saturated aqueous sodium bicarbonate solution (20 mL) and saturated aqueous sodium chloride solution (10 mL) twice, saturated aqueous sodium chloride solution (20 ml), a mixture of HCl (20 mL of 1N HCl) and saturated aqueous sodium chloride solution (10 mL) twice. The organic layer is then dried over magnesium sulfate, filtered, and concentrated. The residue is treated with a mixture of ethyl acetate and tert-butyl methyl ether (1:10, 40 ml) and heated to 50° C.; after stirring for 1 hour at 50° C., it is cooled to 25° C. and stirred overnight. The solid is collected by filtration, dissolved in dichloromethane (10 mL) and filtered through silica gel (20 g); the silica gel is then washed with ethyl acetate (100 mL), 10% methanol in ethyl acetate (200 ml). The combined eluates are concentrated and a mixture of ethyl acetate and tert-butyl methyl ether (5:95, 55 mL) is added. This mixture is heated to 50° C. for 1 h, cooled to 25° C., and stirred overnight, which is then filtered to yield 27b, (MTBE solvate), as a white solid (10.4 g, 75%). ¹H NMR (DMSO-d₆) δ 9.4 (d,1H), 9.03 (d, 1H), 7.65 (s, 1H), 4.96 (ddd, 1H), 4.4 (d,1H), 4.15 (s, 1H), 3.91 (dd, 1H), 3.69 (d, 1H), 3.07 (s, 3H, MTBE), 2.4 (tdd,1H), 1.76-1.65 (m, 2H), 1.57 (dd, 1H), 1.32 (d, 1H), 1.10 (s, 9H, MTBE).

The deuterated compounds 26c and 27c are prepared from the compounds 25 and 5c (Scheme 4c) using the same procedures as described above for the synthesis of the corresponding deuterated compounds 26a, 26b, and 27a, and 27b from the compounds 25, and 5a and 5b (Scheme 4a-b).

### Example 25

### (1R,2S,5S)-N-((S)-1-amino-1-oxo-3-((S)-2-oxopyrrolidin-3-yl-3,4,4,5,5-d₅)propan-2-yl-2,3,3-d3)-3-((S)-3,3-bis(methyl-d₃)-2-(2,2,2-trifluoroacetamido)butanoyl-4,4,4-d₃)-6,6-bis(methyl-d3)-3-azabicyclo[3.1.0]hexane-2-carboxamide (26c)

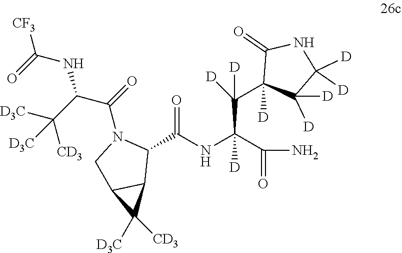

To a stirring solution of 25 (6 g, 13.5 mmol) and the HCl salt of 5c (3.35 g, 15 mol) in 2-butanone (50 mL) is added 2-Hydroxypyridine 1-oxide (0.37 g, 3.4 mmol) and the mixture is cooled to 0° C. N,N-Diisopropylethylamine (DIEA, 7.5 mL) is then added, followed by the addition of EDCI (3 g). The reaction mixture is stirred at room temperature for 16 h, and then diluted with ethyl acetate/tert-butyl methyl ether (1:1, 50 mL) and washed with a mixture of water (20 mL) and saturated aqueous sodium chloride solution (10 mL). The separated organic layer is washed with saturated aqueous sodium chloride solution (30 mL), followed by a mixture of HCl (20 mL of 1M) and saturated aqueous sodium chloride solution (10 mL), then saturated aqueous sodium chloride solution (30 mL), dried over magnesium sulfate, filtered, and organic phase is then concentrated to give 26b as a white solid (7 g,). ¹H NMR (DMSO-d₆) δ 9.4 (d, 1H), 8.29 (d, 1H), 7.5 (s, 1H), 7.3 (br s, 1H), 7 (br s, 1H), 4.4 (d, 1H), 4.35-4.25 (m, 2H), 3.91-3.84 (m, 1H), 3.67 (d, 1H), 2.45-2.34 (m, 1H), 1.70-1.57 (m, 1H), 1.54-1.4 (m, 2H), 1.38 (d, 1H). MS m/z 541.4 [M+1].

### Example 26

### (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-carboxamide (t-butyl methyl ether solvate, MTBE), (27c)

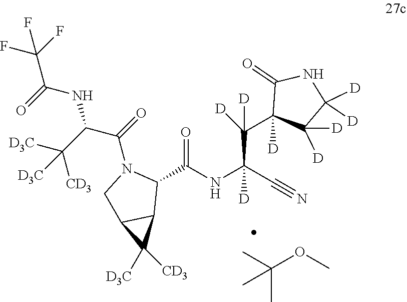

To a solution of 26c (6.1 g, 111. mmol) in dichloromethane (55 mL) is added Methyl N-(triethylammoniosulfonyl)carbamate, inner salt (Burgess reagent, 6.93 g, 27.6 mmol) and the reaction mixture stirred at room temperature for 1 h. A mixture of saturated aqueous sodium bicarbonate solution (20 mL) and saturated aqueous sodium chloride solution (10 mL) is added to the reaction mixture. The organic phase is separated and concentrated. The resulting residue is dissolved in in 50% ethyl acetate/tert-butyl methyl ether (60 mL), washed with a mixture of saturated aqueous sodium bicarbonate solution (20 mL) and saturated aqueous sodium chloride solution (10 mL) twice, saturated aqueous sodium chloride solution (20 mL), a mixture of HCl (20 mL of 1N HCl) and saturated aqueous sodium chloride solution (10 mL) twice. The organic layer is then dried over magnesium sulfate, filtered, and concentrated. The residue is treated with a mixture of ethyl acetate and tert-butyl methyl ether (1:10, 40 mL) and heated to 50° C.; after stirring for 1 hour at 50° C., it is cooled to 25° C. and stirred overnight. The solid is collected by filtration, dissolved in dichloromethane (10 mL) and filtered through silica gel (20 g); the silica gel is then washed with ethyl acetate (100 mL), 10% methanol in ethyl acetate (200 mL). The combined eluates are concentrated and a mixture of ethyl acetate and tert-butyl methyl ether (5:95, 55 mL) is added. This mixture is heated to 50° C. for 1 h, cooled to 25° C., and stirred overnight, which is then filtered to yield 27b, (MTBE solvate), as a white solid (10.4 g, 75%). ¹H NMR (DMSO-d₆) δ 9.4 (d, 1H), 9.03 (d, 1H), 7.65 (s, 1H), 4.96 (ddd, 1H), 4.4 (d, 1H), 4.15 (s,1H), 3.91 (dd,1H), 3.69 (d, 1H), 3.07 (s, 3H, MTBE), 2.4 (tdd,1H), 1.76-1.65 (m, 2H), 1.57 (dd, 1H), 1.32 (d, 1H), 1.10 (s, 9H, MTBE).

### Synthesis of Compounds 28a-c. (Scheme 5)

### Example 27

### (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl)-6,6-bis(methyl-d₃)-3-azabicyclo[3.1.0]hexane-2-carboxamide (28a)

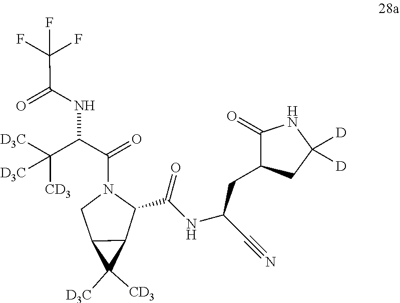 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]

To a round bottom flask containing deuterated compound MTBE solvate 27a (10 g, 16.64 mmol) is added heptane (50 mL) followed by 50 mL of isopropyl acetate (iPrOAc) and the stirring of mixture in the flask is continued overnight at room temperature (Scheme 5). Another 50 mL of heptane is added over 2 h and the mixture is then cooled to 10° C. over a period of half hour and stirred for 3 days. The resulting solid material is filtered, and washed with a mixture of isopropyl acetate (25 mL) and heptane (25 mL). The white solid material is then dried under vacuum at 50° C. to yield the final desired deuterated compound, anhydrous 28a, as a white crystalline solid (7.9 g). ¹H NMR (DMSO-d₆) δ 9.4 (d, 1H), 9 (d, 1H), 7.65 (s, 1H), 4.9 (m, 1H), 4.4 (d, 1H), 4.15 (s, 1H), 3.9 (dd, 1H), 3.7 (d, 1H), 2.40 (m, 1H), 2.14 (m, 1H), 2.11-2.03 (m, 1H), 1.72 (br s, 2H), 1.55 (dd 1H), 1.3 (d, 1H). MS m/z calculated for C₂₃H₁₆D₁₇F₃N₅O₄[M+H]517.24, found 517.24.

### Example 28

### (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-carboxamide (28b)

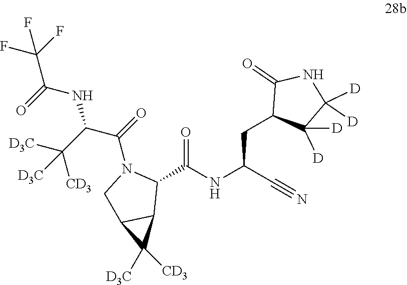 [SMILES: [2H]C([2H])([2H])C1(C([2H])([2H])[2H])[C@@H]2[C@@H](C(=O)N[C@H](C#N)C[C@H]3C(=O)NC([2H])([2H])C3([2H])[2H])N(C(=O)[C@@H](NC(=O)C(F)(F)F)C(C([2H])([2H])[2H])(C([2H])([2H])[2H])C([2H])([2H])[2H])C[C@@H]21]

To a round bottom flask containing deuterated compound 27b (anhydrous MTBE solvate, 10 g, 16.64 mmol) is added heptane (50 mL) followed by 50 ml of isopropyl acetate (iPrOAc) and the stirring of mixture in the flask is continued overnight at room temperature (Scheme 5). Another 50 mL of heptane is added over 2 h and the mixture is then cooled to 10° C. over a period of half hour and stirred for 3 days. The resulting solid material is filtered, and washed with a mixture of isopropyl acetate (25 mL) and heptane (25 mL). The white solid material is then dried under vacuum at 50° C. to yield another deuterated target compound, anhydrous 28b, as a white crystalline solid (8 g). ¹H NMR (DMSO-d₆) δ 9.4 (d,1H), 9 (d,1H), 7.65 (s, 1H), 4.9 (m,1H), 4.4 (d,1H), 4.15 (s, 1H), 3.9 (dd,1H), 3.7 (d,1H), 2.4 (m,1H), 2.15 (m, 1H), 2.11-2.03 (m, 1H), 1.55 (dd, 1H), 1.3 (d,1H). MS m/z calculated for C₂₃H₁₄D₁₉F₃N₅O₄ [M+H]519.24, found 519.24.

### Example 29

### (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-carboxamide (28c)

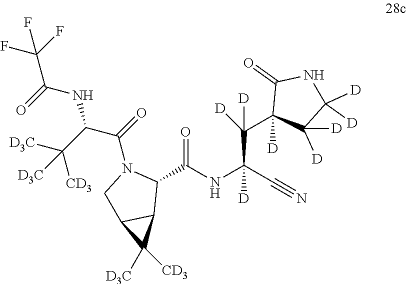

To a round bottom flask containing deuterated compound 27b (anhydrous MTBE solvate, 10 g, 16.64 mmol) is added heptane (50 mL) followed by 50 mL of isopropyl acetate (iPrOAc) and the stirring of mixture in the flask is continued overnight at room temperature (Scheme 5). Another 50 mL of heptane is added over 2 h and the mixture is then cooled to 10° C. over a period of half hour and stirred for 3 days. The resulting solid material is filtered, and washed with a mixture of isopropyl acetate (25 mL) and heptane (25 ml). The white solid material is then dried under vacuum at 50° C. to yield another deuterated target compound, anhydrous 28b, as a white crystalline solid (8 g). ¹H NMR (DMSO-d₆) δ 9.4 (d,1H), 9 (d,1H), 7.65 (s, 1H), 4.9 (m,1H), 4.4 (d,1H), 4.15 (s, 1H), 3.9 (dd,1H), 3.7 (d,1H), 2.4 (m,1H), 2.15 (m, 1H), 2.11-2.03 (m, 1H), 1.55 (dd, 1H), 1.3 (d,1H). MS m/z calculated for C₂₃H₁₀D₂₃F₃N₅O₄C₂₃H₂₅D₈F₃N₅O₄ [M+H]⁺ 523.24, found 523.24.

### Example 30

### Evaluation of Metabolic Stability in CYP3A4 Supersomes™

### Evaluation of Metabolic Stability of Compounds of the Present Invention of Formula I in Human CYP3A4 Supersomes™.

### SUPERSOMES™ Assay.

10 mM stock solutions of test compounds (Compounds of Formula I), are prepared in dimethyl sulfoxide (DMSO). The 10 mM stock solutions are diluted to 15.6 μM in acetonitrile. Human CYP3A4 supersomes™ (1000 pmol/mL, purchased from BD Gentest™ Products and Services) are diluted to 62.5 pmol/mL in 0.1 M potassium phosphate buffer, pH 7.4, containing 3 mM MgCl₂. The diluted supersomes are added to wells of a 96-well polypropylene plate in triplicate. A 10 μL aliquot of the 15.6 μM test compound is added to the supersomes and the mixture is pre-warmed for 10 minutes. Reactions are initiated by addition of pre-warmed NADPH solution. The final reaction volume is 0.5 mL and contained 50 pmol/mL CYP3A4 supersomes™, 0.25 μM test compound (compound of Formula I), and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to 96-well plates which contained 50 μL of ice-cold acetonitrile (ACN) with internal standard to stop the reactions. The plates are stored at 4° C. for 20 minutes after which 100 mL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer.

Data analysis: The in vitro half-lives (t_{1/2} values) for test compounds (compounds of Formula I) are calculated from the slopes of the linear regression of LN (% parent remaining) vs incubation time relationship:

$$in vitro {t}_{1 / 2} = 0.693 / k$$

$$k = - [ slope of line ar regression of \% parent remaining ( In ) vs incubation time ] .$$

### Example 31

### Evaluation of Metabolic Stability in Human Liver Microsomes.

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds (compounds of the present invention of Formula I) are prepared in DMSO. The 7.5 mM stock solutions are diluted to 12.5-50 μM in acetonitrile. The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound (compound of Formula I) is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound (compound of Formula I), and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold acetonitrile with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the corresponding non-deuterated compound of Formula I and the positive control, 7-ethoxycoumarin (1 μM). Testing is done in triplicate.

Data analysis: The in vitro t₂ s for test compounds (compounds of Formula I) are calculated from the slopes of the linear regression of % parent remaining (In) vs incubation time relationship.

$$in vitro {t}_{1 / 2} = \text{.0} \text{.693} / k$$

$$k = - [ slope of line ar regression of \% parent remaining ( In ) vs incubation time ] .$$

### Data Analysis is Performed Using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

Those skilled in the art will recognize or be able to ascertain using no more than routine experimentation, numerous equivalents to the specific reagents can be utilized to produce compounds of the invention. Numerous modifications and variations of the present invention are possible and therefore it is understood that within the scope of the appended claims, the invention may be practiced otherwise that as specifically described herein. Other aspects, advantages and modifications are within the scope of the invention.

### Abbreviations

APCI=atmospheric pressure chemical ionization, AUC=area under the plasma concentration-time curve, Boc=tert-butyloxycarbonyl, CCID=cell culture infective dose, CE=collision energy, CLᵢₙₜ=intrinsic clearance, CLₚ=plasma clearance, CYP=cytochrome P450, Deuterium=D, DCM=dichloromethane, CH₂Cl₂, DIEA=N,N-diisopropylethyl amine, Hunig's base, DMF=N,N-dimethylformamide, DMSO=dimethyl sulfoxide, DTT=dithiothreitol, EDCI=1-[3-(dimethylamino)propyl]-3-ethylcarbodiimide hydrochloride, EDTA=ethylenediaminetetraacetic acid, ESCI=combined electrospray and atmospheric pressure ionization source, ESI=electrospray ionization, EtOAc=ethyl acetate, Fₐ×F_{g}=fraction of the oral dose absorbed, FLIPR=fluorometric imaging plate reader, Oral F=oral bioavailability, GPCR=G-protein coupled receptor, HATU=O-(7-azabenzotriazol-1-yl)-N,N,N′,N′-tetramethyluronium hexafluorophosphate, HEPES=4-(2-hydroxyethyl)-1-piperazineethanesulfonic acid, HLM=human liver microsomes, HOPO=2-hydroxypyridine 1-oxide, HPLC=high performance liquid chromatography, HRMS high resolution mass spectrometry, IPA=isopropanol (iPrOH), IPEA=isopropyl acetate (iPrOAc), iv=intravenous, LCMS=liquid chromatography-mass spectrometry, LC-MS/MS=liquid chromatography tandem mass spectrometry, MeCN=acetonitrile, MeOH=methanol, MTBE=methyl tert-butyl ether, NADPH=nicotinamide adenine dinucleotide phosphate NMR nuclear magnetic resonance, TEA=triethylamine, TFA=trifluoroacetic acid, THF=tetrahydrofuran, TLC=thin layer chromatography.

### Biochemical and Biological Assays

### Determination of Metabolic Stability of Oral SARS-CoV-2 M^{pro} Inhibitors

Stock solutions (7.5 mM) of test compounds are prepared in DMSO. The 7.5 mM stock solutions are diluted to 12.5 μM in acetonitrile. The 20 mg/mL human liver microsomes are diluted to 0.625 mg/mL in 0.1 M potassium phosphate buffer (pH 7.4) containing 3 mM MgCl₂. The diluted microsomes are added to wells of a 96-well deep-well polypropylene plate in triplicate. Ten microliters of the 12.5 μM test compound is added to the microsomes, and the mixture is pre-warmed for 10 min. Reactions are initiated by the addition of pre-warmed NADPH solution. The final reaction volume is 0.5 mL and contained 0.5 mg/mL human liver microsomes, 0.25 μM test compound, and 2 mM NADPH in 0.1 M potassium phosphate buffer (pH 7.4) and 3 mM MgCl₂. The reaction mixtures are incubated at 37° C., and 50-μL aliquots were removed at 0, 5, 10, 20, and 30 min and then added to shallow 96-well plates, which contained 50 μL of ice-cold acetonitrile with internal standard to stop the reactions. The plates are stored at 4° C. for 20 min, after which 100 μL of water is added to the wells of the plate before centrifugation to pellet the precipitated proteins. Supernatants are transferred to another 96-well plate and analyzed for amounts of parent remaining by LC-MS/MS using an Applied Bio-systems API 4000 mass spectrometer. Quantitative analysis by LC-MS/MS is performed using an Applied Bio-systems API 4000 mass spectrometer and utilized an APCI (Atmospheric Pressure Chemical Ionization) source operated in positive ion MRM (multiple reaction monitoring) mode. 7-Ethoxycoumarin (1 μM) is used as a positive control.

### Data Analysis

The in vitro t_{1/2} values for test compounds are calculated from the slopes of the linear regression of the % parent remaining (ln) versus incubation time relationship. Data analysis is performed using Microsoft Excel Software; in vitro t_{1/2}=0.693/k; k=−[slope of linear regression of % parent remaining (ln) vs incubation time].

### In Vitro and In Vivo Pharmacokinetics Activity of Oral SARS-CoV-2 M^{pro} Inhibitors.

Ki values are fit to the Morrison equation with substrate, Km and M^{pro} concentration parameters fixed to values described in the supplemental information.

EC50) values are calculated using data normalized to controls within the assay and fit to a 4 parameter logistic curve fit.

Apparent passive permeability (Papp) from apical to basolateral direction is determined in Madin-Darby canine kidney-ow efflux (MDCK-E) cells L. Di, C. Whitney-Pickett, J. et al. Development of a new permeability assay using low-efflux MDCKII cells. J. Pharm. Sci. 100, 4974-4985 (2011). doi: 10.1002/jps.22674].

Incubations are conducted on a single day in triplicate.
Pharmacokinetic parameters are calculated from plasma concentration-time data; as mean values (n=2-3 male Wistar-Han rats/dosing route).
Oral pharmacokinetics studies are conducted in the fed state. Oral bioavailability (F) is defined as the dose-normalized AUC after oral administration divided by the dose-normalized AUC after intravenous administration.

Test compound (compound of the present invention, Formula I) is orally administered in anhydrous form as well as anhydrous co-solvate form.

The fraction of the oral dose absorbed (Fa×Fg) is estimated using the equation Fa×Fg=F(1−CLblood/Q) [The intestinal first-pass metabolism of substrates of CYP3A4 and P-glycoprotein-quantitative analysis based on information from the literature. Drug Metab. Pharmacokinet. 18, 365-372 (2003). doi: 10.2133/dmpk.18.365].

Coronavirus SARS-CoV-2 M^{Pro} protease (Main protease M^{Pro}) FRET Assay and Analysis

Proteolytic activity of Coronavirus M^{Pro} protease is measured using a continuous fluorescence resonance energy transfer assay. The SARS M^{Pro} FRET assay measures the protease catalyzed cleavage of TAMRA-SITSAVLQSGFRKMK-(DABCYL)-OH to TAMRA-SITSAVLQ and SGFRKMK-(DABCYL)-OH. The fluorescence of the cleaved TAMRA (ex. 558 nm I em. 581 nm) peptide is measured using a TECAN SAFIRE fluorescence plate reader over the course of 10 min. Typical reaction solutions contained 20 mM HEPES (pH 7.0), 1 mM EDTA, 4.0 uM FRET substrate, 4% DMSO and 0.005% Tween-20. Assays are initiated with the addition of 25 nM SARS M^{Pro}. Percent inhibition is determined in duplicate at 0.001 mM level of inhibitor.

Data is analyzed with the non-linear regression analysis program Kalidagraph using the equation:

$$FU = offset + ( limit ) ( 1 - {e}^{\cdot ( kobs ) t} )$$

where offset equals the fluorescence signal of the uncleaved peptide substrate, and limit equals the fluorescence of fully cleaved peptide substrate. The kobs is the first order rate constant for this reaction, and in the absence of any inhibitor represents the utilization of substrate. In an enzyme start reaction which contains an irreversible inhibitors, and where the calculated limit is less than 20% of the theoretical maximum limit, the calculated kobs represents the rate of inactivation of coronavirus main protease (M^{Pro} protease). The slope (kobs/I) of a plot of kobs vs. [I] is a measure of the avidity of the inhibitor for an enzyme. For very fast irreversible inhibitors, kobs/I is calculated from observations at only one or two [I] rather than as a slope.

### Recombinant SARS-CoV-2 M^{pro} Protease Production.

Optimized synthetic genes coding for an Escherichia coli (E. Coli) expression M^{Pro} protease enzyme from the SARS-CoV-2 virus (Wuhan-Hu-1 isolate; accession number MN908947) are designed and ordered from Genscript and IDT/BATJ. Two E. Coli expression constructs are prepared—SARS-CoV-2 M^{Pro} protease (fully mature, authentic) and SARS-CoV-2 M^{Pro}+G (SARS-CoV-2 M^{Pro} protease with an additional Glycine at its N-terminus). The SARS-CoV-2 M^{Pro} construct contains both N and C-terminal His tags. The N-terminal hexa-histidine tag followed with TEV cleavage-site (TTENLYFQ↓SGFRK, arrow indicates the cleavage site), is autocleaved by SARS-CoV-2 M^{Pro} protease during expression to generate the mature N-terminus At the C-terminus, the construct contained a GP hexa-histidine affinity tag, SGVTFQ↓GP, which is a modified PreScission cleavage site that is removed during the purification with PreScission protease. The SARS-CoV-2 M^{Pro}+G construct contains an N-terminal hexa-histidine affinity tag with TEV cleavage-site (TTENLYFQ↓GSGFRK), which is removed during purification with TEV, leaving an extra glycine at the N-terminus E. Coli BL21(DE3) cells harboring the SARS-CoV-2 M^{Pro} expression vector are grown in multiples of 500 ml of LB in 1 liter shake flasks for 5 hours post induction at 16° C. E. Coli BL21(DE3) cells harboring the SARS-CoV-2 M^{Pro}+G expression vector are grown in 6 L of Terrific Broth for 5 h post induction at 30° C. in a high density shake flask. Cell pellets are stored at −80° C. until purification.

### Purification of SARS-CoV-2 M^{pro}

Cell pellets are resuspended and lysed in 50 mM tris(hydroxymethyl)aminomethane (Tris) pH 8, 250 mM NaCl, 10 mM imidazole, 0.25 mM TCEP (Buffer A) via microfluidization and clarified by centrifugation at 29,400 g for 60 min at 4° C. Cleared lysate is added to Ni-probond resin and incubated at 4° C. for 2 h. Ni-resin is loaded on a gravity column and after 15 column volumes washes in Buffer A, protein is eluted in 50 mM Tris pH 8, 250 mM NaCl, 200 mM imidazole, 0.25 mM TCEP. Eluted protein is incubated with TEV/Precision protease and dialyzed overnight. The dialyzed and tag-removed protein is filtered and run over 5 ml nickel column to remove the affinity tag, and the flow through is further purified loading on a Superdex-200 26/60 column equilibrated with 25 mM Tris pH 7.5, 150 mM NaCl, 1 mM EDTA, 1 mM DTT. Pooled fractions are concentrated to 7.10 mg/ml and aliquots are flash-frozen in liquid nitrogen and stored at −80° C. until crystallization.

### Enzyme Kinetics

Test compounds and SARS-CoV-2 M^{Pro} enzyme are incubated at a 1:1 molar ratio of 2 μM compound and 2 μM enzyme in assay buffer (20 mM Tris-HCl, pH 7.3, 100 mM NaCl, 1 mM EDTA, 5 mM TCEP and 0.1% BSA) for 20 minutes. The mixture is then diluted 50-fold into assay buffer followed by a transfer of 5 μL to wells of a black low volume 384-well assay plate. Enzyme activity is monitored on a BMG Pherastar at Ex/Em of 340 nm/460 nm after addition of 5 μL 60 μM peptide substrate (Dabcyl-KTSAVLQ|SGFRKME-Edans) (51) [V. Grum-Tokars, K. Ratia, A. Begaye, S. C. Baker, A. D. Mesecar, Evaluating the 3C-like protease activity of SARS-Coronavirus: recommendations for standardized assays for drug discovery. Virus Res. 133, 63-73 (2008). doi: 10.1016/j.virusres.2007.02.015]. Final reaction conditions are 20 nM enzyme with 20 nM compound and 30 μM peptide substrate. Data is expressed as fraction velocity using DMSO controls with and without M^{Pro} enzyme.

Generation of Assay Ready Plates for Coronavirus M^{Pro} and mammalian protease assay test compound is serially diluted by half-log in 100% DMSO 11 times with a top concentration of 3 mM or serially diluted by 2-fold in 100% DMSO 11 times with a top dose of 0.1 mM. A volume of 300 nL of each dilution was spotted into a separate plate ready for enzyme and substrate additions. The top dose of compound in the assay was 30 M or 1 M with the final DMSO concentration at 1%.

### Biochemical Determination for Human Coronavirus M^{pro} Assays

The respective human coronavirus M^{Pro} in assay buffer (20 mM Tris-HCl, pH 7.3, 100 mM NaCl, 1 mM EDTA, 5 mM TCEP) and 0.1% BSA is added to assay-ready plates containing test compound (compound the present invention, Formula I). The enzymatic reaction is then immediately initiated with the addition of 5 μL substrate in assay buffer. Initial rates are measured by following the fluorescence of the cleaved substrate using a Spectramax (Molecular Devices) fluorescence plate reader in the kinetic format.

Test compounds (compounds of the present invention, compounds of Formula I) are tested at final concentrations up to 30 M in 1% DMSO and compared to the broad-spectrum antiviral compound GC376 [Y. Kim, S. Lovell, et al, Broad-spectrum antivirals against 3C or 3C-like proteases of picornaviruses, noroviruses, and coronaviruses. J. Virol. 86, 11754-11762 (2012). doi: 10.1128/JVI.01348-12] which produced 100% inhibition at 30 μM. Control wells (0% inhibition) contained 1% DMSO with substrate and protease and did not contain compound. The reaction is allowed to progress for 60 minutes at 23° C. after which the plate is read on a Molecular Devices Spectramax M2e reader at an Ex/Em of 340 nm/490 nm.

### Mammalian Protease Panel

The respective protease in assay buffer (50 mM Tris with 100 mM sodium chloride and Brij 35 at pH 8.0 except for cathepsin D pH 3.5 and HIV pH 5.5) is added to assay ready compound plates. The cathepsin L buffer is 400 mM sodium acetate pH 5.5 with 4 mM EDTA and 8 mM DTT. The enzymatic reaction is initiated with the addition of indicated substrate in assay buffer and proceeded at room temperature for 2 h. Final DMSO concentration is below 1%. Initial rates are measured by following the fluorescence of the cleaved substrate using a Spectramax (Molecular Devices) fluorescence plate reader in the kinetic format.

### Data Analysis for Mammalian and Coronavirus Protease Panels.

Percent inhibition values are calculated based on control wells containing DMSO only (0% inhibition) and wells containing a control compound (100% inhibition). IC₅₀ values are generated based on a four-parameter logistic fit model using ActivityBase software (IDBS). Percent activity values are calculated based on control wells containing no compound (100% activity) and wells containing the broad-spectrum antiviral compound GC376 (0% activity). Percent activity values are calculated based on control wells containing no compound (100% activity) and wells containing an internal control compound (0% activity). Ki values are fit to the tight binding Morrison equation with fixed parameters for enzyme concentration, substrate concentration and the Km parameter using ActivityBase software (IDBS) indicated below

$$TB K𝔦 : {v}_{i} = b + v 0 * ( 1 - \frac{2 * [ I ]}{E + [ I ] + Ki * \frac{[ S ] + Km}{Km} + \sqrt{{( E + [ I ] + Ki * \frac{[ S ] + Km}{Km} )}^{2}} - 4 E [ I ]} ) = function ( b , v 0 , Ki , E )$$

### Cellular Antiviral Activity

The ability of compounds to inhibit viral induced cytopathic effect (CPE) against human coronaviruses (SARS-CoV-2, SARS-CoV-1, hCoV-229E, MERS-CoV) is assessed by monitoring cell viability using two different assay endpoints in VeroE6, MRC-5 or Vero81 cells.

VeroE6 cells that are enriched for hACE2 expression are batched innoculated with SARS-CoV-2 (USA_WA1/2020) at a multiplicity of infection (MOI) of 0.002 in a BSL-3 lab (Southern Research Institute). Virus innoculated cells are then added to assay ready compound plates at a density of 4,000 cells/well in DMEM containing 2% heat inactivated FBS. Cells were incubated for 3 days at 37° C. with 5% CO2, a time at which virus induced CPE is 95% in the untreated, infected control conditions.

MRC-5 cells at a density of 20,000 cells/well are incubated overnight in MEM containing 5% FBS at 37° C. and 5% CO₂. Following addition of test compounds, HCoV-229E virus (ATCC VR-740) (200 TCID50) is added at concentrations which correspond to a multiplicity of infection (MOI) of 0.0007 to MRC-5 cells. Cells are incubated for 3 days at 35° C. with 5% CO2.

Vero81 (ATCC CCL-81) cells are batched inoculated with MERS (EMC/2012) at M.O.I. ˜0.01 in a BSL-3 lab. Virus inoculated cells are then added to assay ready compound plates at a density of 4,000 cells/well in DMEM containing 2% heat inactivated FBS. Following a 4 day incubation at 37° C. with 5% CO₂, a time at which virus-induced CPE is 90 to 95% in the untreated, infected control conditions.

MRC-5 cells at a density of 20,000 cells/well are incubated overnight in MEM containing 5% FBS at 37° C. and 5% CO₂. Following addition of test compounds, HCoV-229E virus (ATCC VR-740) (200 TCID50) is added at concentrations which correspond to a multiplicity of infection (MOI) of 0.0007 to MRC-5 cells. Cells are incubated for 3 days at 35° C. with 5% CO₂.

Vero81 (ATCC CCL-81) cells are batched inoculated with MERS (EMC/2012) at M.O.I. ˜0.01 in a BSL-3 lab (Southern Research Institute). Virus inoculated cells are then added to assay ready compound plates at a density of 4,000 cells/well in DMEM containing 2% heat inactivated FBS. Following a 4 day incubation at 37° C. with 5% CO₂, a time at which virus-induced CPE is 90 to 95% in the untreated, infected control conditions.

Cell viability is evaluated using Cell Titer-Glo (Promega), according to the manufacturer's protocol, which quantitates ATP levels. Cytotoxicity of compounds is assessed in parallel in assay ready plates with non-infected cells in a BSL-2 lab.

Test compound/s _{(compounds of the present invention, compounds of Formula I)} are tested either alone or in the presence of the P-glycoprotein (P-gp) inhibitor. The inclusion of P-gp is to assess if the test compound(s) are being effluxed out of cells due to endogenous expression of P-glycoprotein in the cell line. Percent effect at each concentration of test compound is calculated based on the values for the no virus control wells and virus containing control wells on each assay plate. The concentration required for a 50% response (EC₅₀) value is determined from these data using a 4-parameter logistic model. EC₅₀ curves are fit to a Hill slope of 3 when ≥3 and the top dose achieved ≥50% effect. If cytotoxicity is detected at greater than 30% effect, the corresponding concentration data is eliminated from the EC₅₀ determination.

For cytotoxicity plates, a percent effect at each concentration of test compound is calculated based on the values for the cell only control wells and hyamine or no cell containing control wells on each assay plate. The CC₅₀ value is calculated using a 4-parameter logistic model. A therapeutic index is then calculated by dividing the CC₅₀ value by the EC₅₀ value.

Antiviral activity of test compounds is evaluated in differentiated normal human bronchial epithelial (dNHBE) cells in a BSL-3 facility. The dNHBE cells (EpiAirway) are procured from MatTek Corporation (Ashland, MA) and are grown on trans-well inserts consisting of approximately 1.2×10⁶ cells in MatTek's proprietary culture medium (AIR-100-MM) added to the basolateral side, with the apical side exposed to a humidified 5% CO₂ environment at 37° C. On day 1, dNHBE cells are infected with SARS-CoV-2 strain USA-WA1/2020 at a MOI of approximately 0.0015 CCID50 per cell, and test compound treatment is carried out by inclusion of drug dilutions in basolateral culture media. At day 3 and day 5, virus released into the apical compartment is harvested by the addition of 0.4 ml culture media. The virus titer is then quantified by infecting Vero76 cells in a standard endpoint dilution assay and virus dose that is able to infect 50% of the cell cultures (CCID50 per ml) is calculated [L. J. Reed, H. Muench, A simple method of estimating fifty percent endpoints. Am. J. Hygiene27, 493 497 (1938). doi: 10.1093/oxfordjournals.aje.a118408]. To determine the EC₅₀ and EC₉₀, the CCID50/ml values are normalized to that of no drug control as a percentage of inhibition and plotted against compound concentration in GraphPad Prism software by using four-parameter logistic regression.

### Mouse-Adapted SARS-CoV-2 Infection and Treatment Studies

The in vivo infection studies are performed in an animal biosafety level 3 (ABSL3) facility in the AAALAC-accredited Laboratory Animal Research Center. Pharmacokinetics studies are performed in an animal biosafety level 2 (ABSL2) facility. The study procedures are conducted with approval by the Institutional Animal Care and Use Committee.

A total of 24 BALB/c mice (Charles River, 8 week old female, n=6 mice/group) are divided into 4 groups: group 1: untreated, infected control; group 2: 300 mg/kg of test compound(s); group 3: 1000 mg/kg of test compound(s) and group 4: untreated, uninfected control (for pharmacokinetic analysis and normal weight). Mice are anesthetized by intraperitoneal (i.p.) injection of ketamine/xylazine (50 mg/kg/5 mg/kg) and inoculated intranasally (i.n.) with 1×10⁵ 50% cell culture infectious dose (CCID50) of SARS-CoV-2 MA10 (90 ml/nares). The mouse-adapted MA10 virus (30)) can be obtained fromauthorized and approved laboratories. For oral (p.o.) administration, test compound(s) are solubilized in 0.5% methylcellulose in water, containing 2% Tween80. Mice are dosed twice daily (BID)×4 days beginning at 4 hours post infection. Mice are weighed daily starting at day 0 until end of study to measure infection-associated weight loss. At 4 days post infection (dpi), mice are euthanized by isoflurane inhalation. The lungs are collected and placed in 1 ml PBS and stored at −80° C. for evaluation of lung virus titers or collected for histopathology as described below. For virus titer assays, serial log 10 dilutions of 1.0 ml lung tissue homogenates are performed in quadruplicate on confluent monolayers of Vero 76 cells seeded in 96-well microplates. The cells are incubated at 37° C. and 5% CO2 for 6 days and then scored for cytopathic effect (CPE) using a light microscope. Virus lung titer (CCID50/ml (Log10) is calculated by linear regression using the Reed-Muench method [L. J. Reed, H. Muench, A simple method of estimating fifty percent endpoints. Am. J. Hygiene27, 493-497 (1938). doi: 10.1093/oxfordjournals.aje.a118408].

### Metabolic Stability of Compounds in HLM

Substrate stocks (30 mM) are prepared in DMSO and diluted to 100-times the incubation concentration in 50% water 50% acetonitrile, for a final organic composition of 0.489% acetonitrile and 0.00334% DMSO in the incubations. Substrate (0.1 or 1 μM) is incubated in HLM (1 mg/ml) diluted in potassium phosphate buffer (100 mM, pH 7.4) supplemented with MgCl₂ (3.3 mM) and NADPH (1.3 mM) in a final volume of 300 μl. Test Compound(s) incubations were conducted with 2 mg/ml human liver microsomes. Incubations are conducted at 37° C. open to ambient air. A no-NADPH control was carried out in parallel. Incubations were conducted in triplicate. At various time points (typically 0.25, 2, 4, 6, 10, 20, 40 and 60 min), a 20 μl aliquot of incubate is removed and quenched in 100 μl of acetonitrile containing internal standard indomethacin (50 ng/ml). Samples are vortexed, centrifuged (5 min, 2300× g) and clean supernatant is diluted with an equal volume of water containing 0.2% formic acid. Samples are directly analyzed by liquid chromatography tandem mass spectrometry (LC-MS/MS). Analyst software (Sciex, Framingham, MA) is used to measure peak areas. Peak area ratios of analyte to internal standard are calculated. Substrate depletion half-life (t_{1/2}) and intrinsic clearance (CLint) are calculated. using E-WorkBook v10 (ID Business Solutions, Guildford, Surrey, UK). The natural log of peak area ratios versus time were fitted using linear regression, the slope of which (k) was converted to t1/2 values, where t1/2=−0.693/k. To estimate in vitro CLint in HLM, the t1/2 for substrate depletion was scaled using the following equation [R. S. Obach, et al. The prediction of human pharmacokinetic parameters from preclinical and in vitro metabolism data. J. Pharmacol. Exp. Ther. 283, 46-58 (1997)].

$${CL}_{int , app} = \frac{0 \text{.693} \cdot ml incubation}{{t}_{\frac{1}{2}} \cdot amount of protein in incubation}$$

The incubation volume is 0.3 ml and the protein density is typically 1 mg liver microsomes/mL.

### Effect of Cytochrome P450 (CYP)3A4 Inhibitors on Test Compounds in HLM.

Incubations are conducted in 100 mM potassium phosphate buffer (pH 7.4) containing MgCl₂ (3.3 mM), NADPH (1.3 mM), HLM (1 mg/ml), and deuterated compounds of the invention e.g. 28 a-c) at 37° C. for 0-80 min in the absence or presence of selective CYP3A inhibitor/s (1 μM). Incubations in the absence of NADPH are incubated for 0-60 min. The incubation volume is 0.4 ml. Reactions are terminated by transferring an aliquot (40 μl) of the incubation mixture to a solution of acetonitrile containing internal standard (25 ng/ml diclofenac, 160 μl). Quenched samples are centrifuged (2300×g) for 5 min, followed by the transfer of supernatant (100 l) to 96-well plates with 100 μl of H₂O added to the samples. Incubations in the presence of NADPH are conducted in triplicate. Incubations in the absence of NADPH are conducted in duplicate. Samples are analyzed by LC-MS/MS for remaining test compounds and CLint is estimated as described above.

Test Compound/s (compounds of the present invention, compounds of Formula I) (10 μM) are incubated in pooled (mixed gender of 50) HLM (protein concentration=2 mg/mL) and recombinant human CYP enzymes (CYP1A1, CYP1A2, CYP1B1, CYP2A6, CYP2B6, CYP2C8, CYP2C9, CYP2C18, CYP2C19, CYP2D6, CYP3A4, CYP3A5, CYP3A7, CYP2E1, CYP2J2, CYP4F2, 100 pmol/mL each) in 100 mM phosphate buffer (pH 7.4) containing NADPH (1.3 mM), MgCl2 (3.3 mM) at 37° C. Incubations are carried out for 1 h and quenched with the addition of acetonitrile (0.6 ml). A second set of incubation mixtures are prepared in which test compound(s) only added after quenching. These incubations served as controls to assess metabolite formation. The terminated incubation mixtures are centrifuged (1800×g) for 5 min and the supernatants (0.6 ml) are evaporated to dryness using a Genevac evaporative centrifuge, and the residue is reconstituted in 50 μl of 1% formic acid containing 20% acetonitrile for analysis by LCMS analysis. Reconstituted samples are analyzed and operated in positive ion mode. Injection volumes are 5 to 15 μl, depending on the experimental run. A commercially available chromatography column (for example from Agilent, Santa Clara, California) is used (2.1×100 mm, 2.6 μm) with a flow rate of 0.4 ml/min heated to 45° C. Mobile phase A is comprised of 0.1% formic acid in water and mobile phase B is comprised of acetonitrile. The gradient system comprised of: initially, 5% B held for 0.5 min followed by a linear gradient to 70% B at 11 min, a second linear gradient to 95% B at 13 min, a 1 min wash at 95% B, and finally a 2 min re-equilibration period at 5% B. UV is monitored between 200 to 400 nm and chromatograms are reconstructed using k at 200 nm. Mass spectral data are collected in positive ion mode.

### Activity of Compounds

Compound IC₅₀ values are determined using 10-pt concentration ranges (10 μM-0.005 μM or 100 nM-0.005 nM). Compounds are dissolved in 100% DMSO with the final DMSO concentration in each assay ·0.7%. Rates of substrate hydrolysis are determined from time courses and converted to percent (%) inhibition. Percent inhibition is plotted versus compound concentration and fit to the four parameter logistic equation by non-linear regression using Prism Graphpad software (Graphpad Software, Inc., San Diego, CA.)

Using the methodology described above, representative compounds of this invention are evaluated and found to exhibit IC₅₀ values of (<100 nM) thereby demonstrating and confirming the utility of the compounds of this invention as effective SARS(COV-2), SARS(COV) and MERS(Cov) inhibitors.

Accordingly, the novel compounds of the present invention are useful in human therapy for treating the unprecedented fatal pandemic disease Covid-19 caused by SARS-CoV-2. These novel drug candidates also has potential for treating the severe acute respiratory syndrome caused by the virus (SARS-CoV), Middle East Respiratory Syndrome (MERS) by administration to a patient in need of such treatment of a therapeutically effective amount of compounds of this invention and salts thereof.

In the treatment of SARS-CoV-2 (Covid-19), the compounds of this invention may be utilized in compositions such as tablets, capsules, injectables or elixirs for oral administration, suppositories for rectal administration, sterile solutions or suspensions for parenteral or intramuscular administration, and the like. The compounds of this invention can be administered to patients (humans and animals) in need of such treatment in dosages that will provide optimal pharmaceutical efficacy. Although the dose will vary from patient to patient depending upon the nature and severity of disease, the patient's weight, special diets then being followed by a patient, concurrent medication, and other factors which those skilled in the art will recognize, the dosage range will generally be appropriately selected which can be administered in single or multiple doses.

The compounds of this invention can also be administered alone and in combination with other drugs including the following drugs, protease inhibitor, Camostat mesylate, antimalarial drugs including Chloroquine,and hydroxycholoroqine, viral RNA-dependent RNA polymerase drugs including Remdesivir, Ribavirin, Pimodivir, Penciclover, Galidisivir, Favipiravir, and Baloxavir marboxil, and antiviral drug Baricitinib and viral protease inhibitors including Darunavir, Lopinavir, Ritonavir, and viral protein expression drug Nitazoxanide, and viral surface protein drug Umifenovir. and ribonucloside analogs for example EIDD-2801; comprising a therapeutically effective amount of the novel compound of this invention together with a pharmaceutically acceptable carrier therefor.

A compound or mixture of compounds of Formula I or a physiologically acceptable salt is compounded with a physiologically acceptable vehicle, carrier, excipient, binder, preservative, stabilizer, flavor etc., in a unit dosage from as called for by accepted pharmaceutical practice. The amount of active substance in these compositions or preparations is such that a suitable dosage in the range indicated is obtained.

Illustrative of the adjuvants which can be incorporated in tablets, capsules and the like are the following: a binder such as gum tragacanth, acacia, corn starch or gelatin; an excipient such as microcrytalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. When the dosage unit form is capsule, it may contain, in addition to materials of the above type, a liquid carrier such as fatty oil. Various other materials may be present as coatings or to otherwise modify the physical form of the dosage limit.

Sterile compositions for injection can be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like can be incorporated as required.

What is claimed is:

1. A deuterium enriched compound of chemical structural formula I, 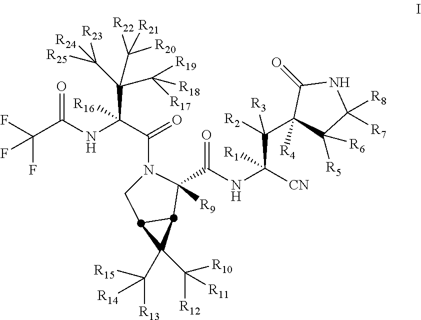
and diastereomers, enantiomers, or a pharmaceutically acceptable salt thereof, wherein,
R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, are independently D (deuterium), or H (hydrogen).

2. The deuterium-enriched compounds of claim 1 of chemical structural formula I selected from the group consisting of:
(i) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-carboxamide, 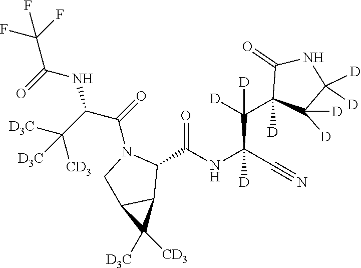
(ii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-carboxamide], 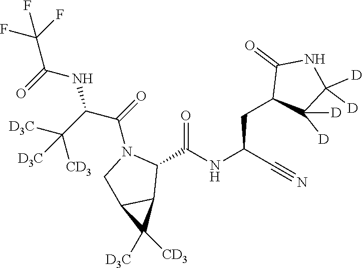 [SMILES: [2H]C([2H])([2H])C1(C([2H])([2H])[2H])[C@@H]2[C@@H](C(=O)N[C@H](C#N)C[C@H]3C(=O)NC([2H])([2H])C3([2H])[2H])N(C(=O)[C@@H](NC(=O)C(F)(F)F)C(C([2H])([2H])[2H])(C([2H])([2H])[2H])C([2H])([2H])[2H])C[C@@H]21]
(iii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl)-6,6-bis(methyl-d₃)-3-azabicyclo[3.1.0]hexane-2-carboxamide], 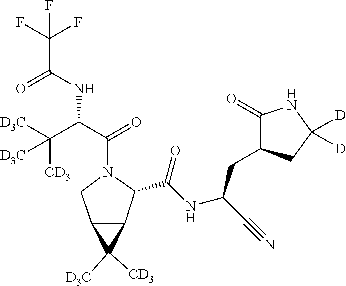 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(iv) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-d₁-2-carboxamide], 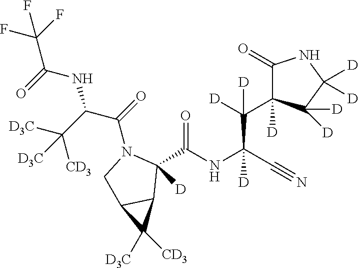
(v) (1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-carboxamide], 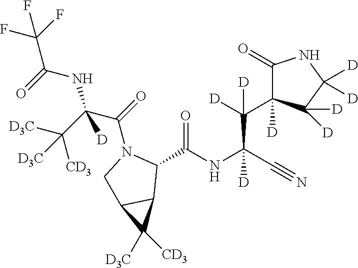
(vi) (1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-d-2-carboxamide], 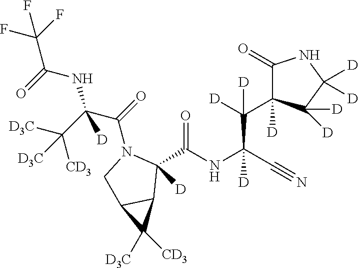
(vii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl)-6,6-bis(methyl-d₃)-3-azabicyclo[3.1.0]hexane-2-carboxamide], 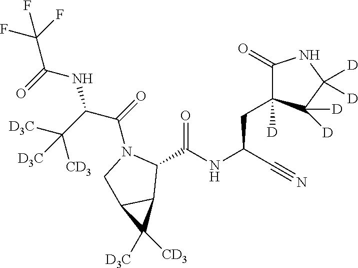 [SMILES: [2H]C([2H])([2H])C1(C([2H])([2H])[2H])[C@@H]2[C@@H](C(=O)N[C@H](C#N)C[C@@]3([2H])C(=O)NC([2H])([2H])C3([2H])[2H])N(C(=O)[C@@H](NC(=O)C(F)(F)F)C(C([2H])([2H])[2H])(C([2H])([2H])[2H])C([2H])([2H])[2H])C[C@@H]21]
(viii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-d₁-2-carboxamide], 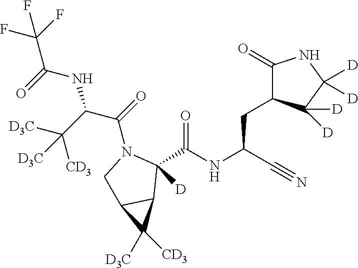 [SMILES: [2H]C([2H])([2H])C1(C([2H])([2H])[2H])[C@H]2[C@@H]1CN(C(=O)[C@@H](NC(=O)C(F)(F)F)C(C([2H])([2H])[2H])(C([2H])([2H])[2H])C([2H])([2H])[2H])[C@]2([2H])C(=O)N[C@H](C#N)C[C@H]1C(=O)NC([2H])([2H])C1([2H])[2H]]
(ix) (1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-carboxamide], 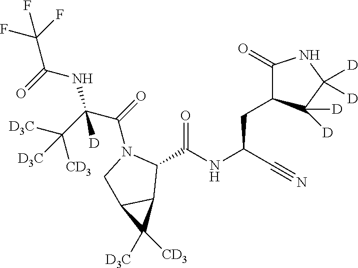
(x) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl)-6,6-bis(methyl-d₃)-3-azabicyclo[3.1.0]hexane-2-d-2-carboxamide], 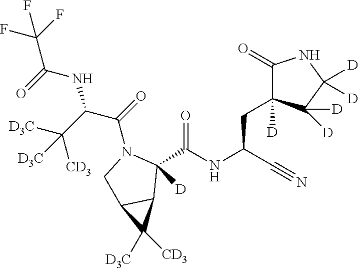 [SMILES: [2H]C([2H])([2H])C1(C([2H])([2H])[2H])[C@H]2[C@@H]1CN(C(=O)[C@@H](NC(=O)C(F)(F)F)C(C([2H])([2H])[2H])(C([2H])([2H])[2H])C([2H])([2H])[2H])[C@]2([2H])C(=O)N[C@H](C#N)C[C@@]1([2H])C(=O)NC([2H])([2H])C1([2H])[2H]]
(xi) (1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,4,4,5,5-d₅)ethyl)-6,6-bis(methyl-d₃)-3-azabicyclo[3.1.0]hexane-2-d₁-2-carboxamide], 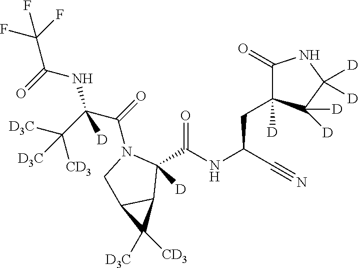 [SMILES: [2H]C([2H])([2H])C1(C([2H])([2H])[2H])[C@H]2[C@@H]1CN(C(=O)[C@]([2H])(NC(=O)C(F)(F)F)C(C([2H])([2H])[2H])(C([2H])([2H])[2H])C([2H])([2H])[2H])[C@]2([2H])C(=O)N[C@H](C#N)C[C@@]1([2H])C(=O)NC([2H])([2H])C1([2H])[2H]]
(xii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl)-6,6-bis(methyl-d₃)-3-azabicyclo[3.1.0]hexane-2-carboxamide], 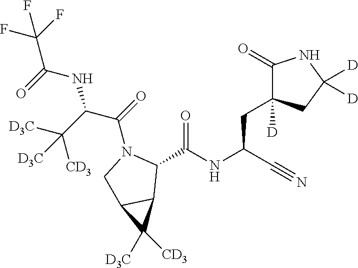 [SMILES: [2H]C1([2H])C[C@@]([2H])(C[C@@H](C#N)NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(xiii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl)-6,6-bis(methyl-d₃)-3-azabicyclo[3.1.0]hexane-2-d-2-carboxamide], 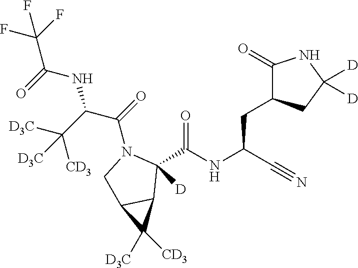 [SMILES: [2H]C1([2H])C[C@@H](C[C@@H](C#N)NC(=O)[C@]2([2H])[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(xiv) (1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-5,5-d₂)ethyl)-6,6-bis(methyl-d₃)-3-azabicyclo[3.1.0]hexane-2-carboxamide], 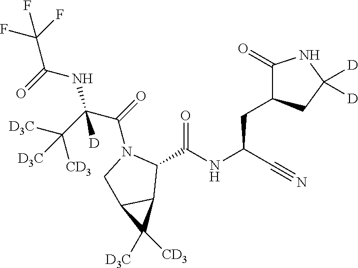
(xv) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl)-6,6-bis(methyl-d₃)-3-azabicyclo[3.1.0]hexane-2-d₁-2-carboxamide], 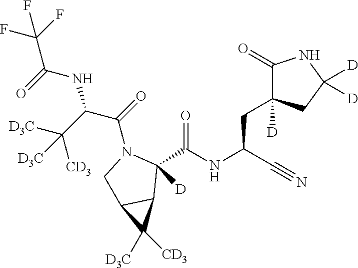 [SMILES: [2H]C1([2H])C[C@@]([2H])(C[C@@H](C#N)NC(=O)[C@]2([2H])[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(xvi) (1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,5,5-d₃)ethyl)-6,6-bis(methyl-d₃)-3-azabicyclo[3.1.0]hexane-2-d-2-carboxamide], 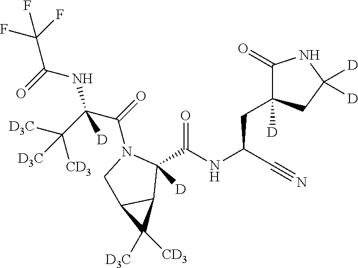 [SMILES: [2H]C1([2H])C[C@@]([2H])(C[C@@H](C#N)NC(=O)[C@]2([2H])[C@@H]3[C@H](CN2C(=O)[C@]([2H])(NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(xvii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl-1-d)-6,6-bis(methyl-d₃)-3-azabicyclo[3.1.0]hexane-2-carboxamide], 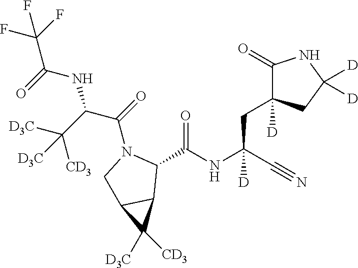
(xviii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl-1-d)-6,6-bis(methyl-d₃)-3-azabicyclo[3.1.0]hexane-2-d-2-carboxamide], 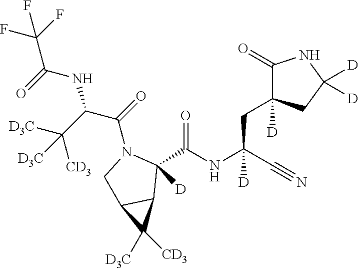
(xix) (1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,5,5-d₃)ethyl-1-d)-6,6-bis(methyl-d₃)-3-azabicyclo[3.1.0]hexane-2-d₁-2-carboxamide], 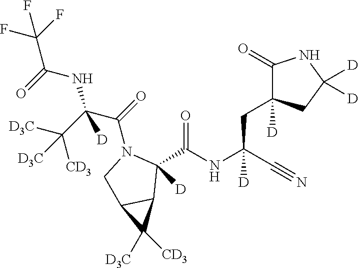
(xx) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl-2,2-d₂)-6,6-bis(methyl-d₃)-3-azabicyclo[3.1.0]hexane-2-carboxamide], 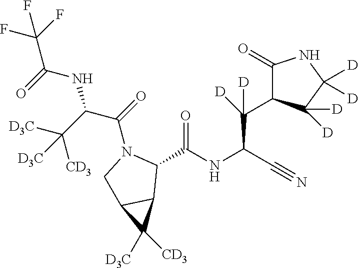 [SMILES: [2H]C([2H])([C@H]1C(=O)NC([2H])([2H])C1([2H])[2H])[C@@H](C#N)NC(=O)[C@@H]1[C@@H]2[C@H](CN1C(=O)[C@@H](NC(=O)C(F)(F)F)C(C([2H])([2H])[2H])(C([2H])([2H])[2H])C([2H])([2H])[2H])C2(C([2H])([2H])[2H])C([2H])([2H])[2H]]
(xxi) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl-2,2-d₂)-6,6-bis(methyl-d₃)-3-azabicyclo[3.1.0]hexane-2-carboxamide], 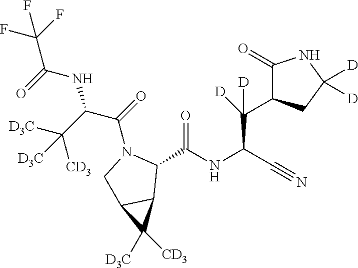 [SMILES: [2H]C1([2H])C[C@@H](C([2H])([2H])[C@@H](C#N)NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(xxii) (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4-d₂)ethyl-2,2-d₂)-6,6-bis(methyl-d₃)-3-azabicyclo[3.1.0]hexane-2-carboxamide], 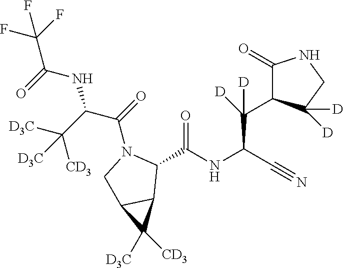 [SMILES: [2H]C1([2H])CNC(=O)[C@@H]1C([2H])([2H])[C@@H](C#N)NC(=O)[C@@H]1[C@@H]2[C@H](CN1C(=O)[C@@H](NC(=O)C(F)(F)F)C(C([2H])([2H])[2H])(C([2H])([2H])[2H])C([2H])([2H])[2H])C2(C([2H])([2H])[2H])C([2H])([2H])[2H]]
or a pharmaceutically acceptable salt thereof.

3. A method of treating a coronavirus infection comprising administering a pharmaceutically effective dose of a compound selected from a deuterium enriched compound of chemical structural formula I, 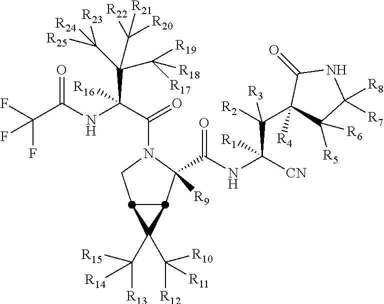
and diastereomers, enantiomers, or a pharmaceutically acceptable salt thereof, wherein, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, are independently selected from D (deuterium), and H (hydrogen) or a pharmaceutically acceptable salt thereof with a pharmaceutically effective dilutant or carrier and said coronavirus infection is selected from COVID-19, (SARS-CoV)-2, (SARS-CoV), and MERS-CoV.

4. The method of claim 3 wherein the compound is selected from the group consisting of (1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-carboxamide, 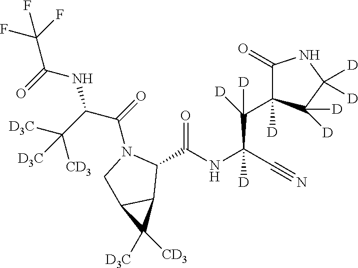
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-carboxamide], 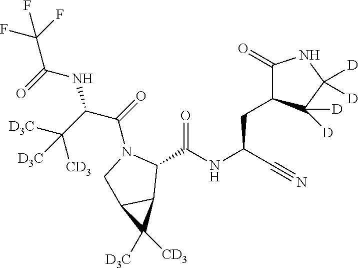 [SMILES: [2H]C([2H])([2H])C1(C([2H])([2H])[2H])[C@@H]2[C@@H](C(=O)N[C@H](C#N)C[C@H]3C(=O)NC([2H])([2H])C3([2H])[2H])N(C(=O)[C@@H](NC(=O)C(F)(F)F)C(C([2H])([2H])[2H])(C([2H])([2H])[2H])C([2H])([2H])[2H])C[C@@H]21]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl)-6,6-bis(methyl-d₃)-3-azabicyclo[3.1.0]hexane-2-carboxamide], 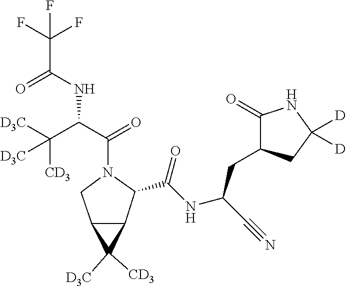 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-d₁-2-carboxamide], 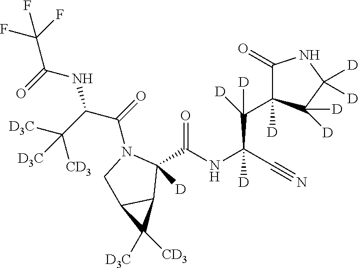
(1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,4,4,5,5-d₅)ethyl-1,2,2-d₃)-6,6-bis(methyl-d₃)-3-azabicyclo[3.1.0]hexane-2-carboxamide], 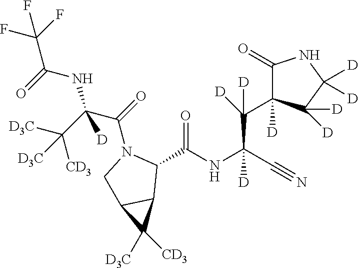
(1R,2S,5S)-3,3-bis(methyl-d₃)-2-(2,2,2-trifluroacetamido)butanoyl-2,4,4,4-d₄)-N-((S)-1-cyano-2-((S)-2-oxopyroolidin-3-yl-3,4,4,5,5-d5)ethyl-1,2,2-d₃)-6,6-bis(methyl-d₃)-3-azabicyclo[3.1.0]hexane-2-d-2-carboxamide], 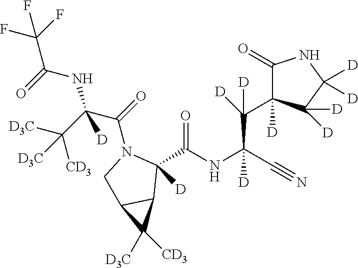
(1R,2S,5S)-3-((S)-3,3-bis(methyl-d₃)-2-(2,2,2-trifluoroacetamido)butanoyl-4,4,4-d₄)-N-((S)-1-cyano-2-((S)-2-oxopyrrolidin-3-yl-3,4,4,5,5-d₅)ethyl)-6,6-bis(methyl-d₃)-3-azabicyclo[3.1.0]hexane-2d-2-carboxamide], 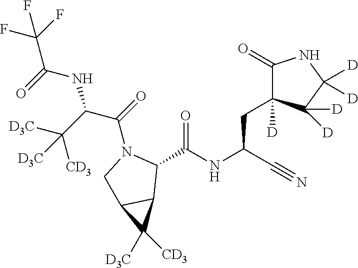 [SMILES: [2H]C([2H])([2H])C1(C([2H])([2H])[2H])[C@@H]2[C@@H](C(=O)N[C@H](C#N)C[C@@]3([2H])C(=O)NC([2H])([2H])C3([2H])[2H])N(C(=O)[C@@H](NC(=O)C(F)(F)F)C(C([2H])([2H])[2H])(C([2H])([2H])[2H])C([2H])([2H])[2H])C[C@@H]21]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-d₁-2-carboxamide], 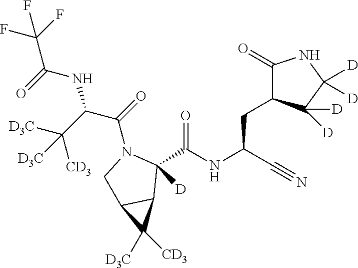 [SMILES: [2H]C([2H])([2H])C1(C([2H])([2H])[2H])[C@H]2[C@@H]1CN(C(=O)[C@@H](NC(=O)C(F)(F)F)C(C([2H])([2H])[2H])(C([2H])([2H])[2H])C([2H])([2H])[2H])[C@]2([2H])C(=O)N[C@H](C#N)C[C@H]1C(=O)NC([2H])([2H])C1([2H])[2H]]
(1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-4,4,5,5-d₄)ethyl)-6,6-bis(methyl-d₃)-3-azabicyclo[3.1.0]hexane-2-carboxamide], 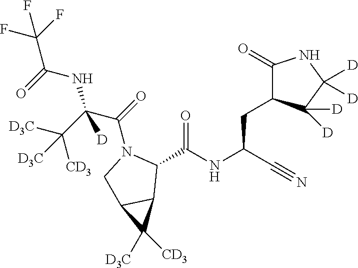 [SMILES: [2H]C([2H])([2H])C1(C([2H])([2H])[2H])[C@@H]2[C@@H](C(=O)N[C@H](C#N)C[C@H]3C(=O)NC([2H])([2H])C3([2H])[2H])N(C(=O)[C@]([2H])(NC(=O)C(F)(F)F)C(C([2H])([2H])[2H])(C([2H])([2H])[2H])C([2H])([2H])[2H])C[C@@H]21]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,4,4,5,5-d₅)ethyl)-6,6-bis(methyl-d₃)-3-azabicyclo[3.1.0]hexane-2-d-2-carboxamide], 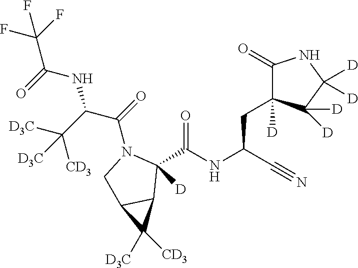 [SMILES: [2H]C([2H])([2H])C1(C([2H])([2H])[2H])[C@H]2[C@@H]1CN(C(=O)[C@@H](NC(=O)C(F)(F)F)C(C([2H])([2H])[2H])(C([2H])([2H])[2H])C([2H])([2H])[2H])[C@]2([2H])C(=O)N[C@H](C#N)C[C@@]1([2H])C(=O)NC([2H])([2H])C1([2H])[2H]]
(1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,4,4,5,5-d₅)ethyl)-6,6-bis(methyl-d₃)-3-azabicyclo[3.1.0]hexane-2-d₁-2-carboxamide], 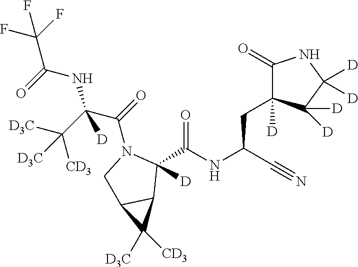 [SMILES: [2H]C([2H])([2H])C1(C([2H])([2H])[2H])[C@H]2[C@@H]1CN(C(=O)[C@]([2H])(NC(=O)C(F)(F)F)C(C([2H])([2H])[2H])(C([2H])([2H])[2H])C([2H])([2H])[2H])[C@]2([2H])C(=O)N[C@H](C#N)C[C@@]1([2H])C(=O)NC([2H])([2H])C1([2H])[2H]]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl)-6,6-bis(methyl-d₃)-3-azabicyclo[3.1.0]hexane-2-carboxamide], 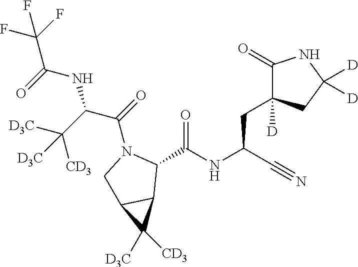 [SMILES: [2H]C1([2H])C[C@@]([2H])(C[C@@H](C#N)NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl)-6,6-bis(methyl-d₃)-3-azabicyclo[3.1.0]hexane-2-d-2-carboxamide], 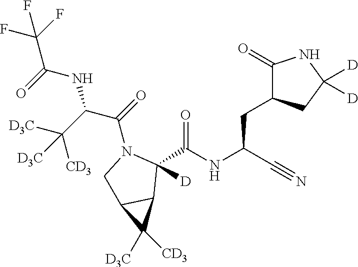 [SMILES: [2H]C1([2H])C[C@@H](C[C@@H](C#N)NC(=O)[C@]2([2H])[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-5,5-d₂)ethyl)-6,6-bis(methyl-d₃)-3-azabicyclo[3.1.0]hexane-2-carboxamide], 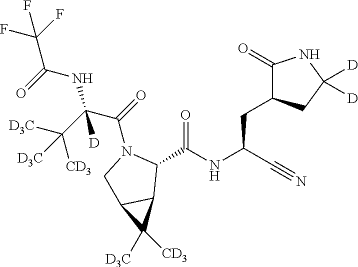
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl)-6,6-bis(methyl-d₃)-3-azabicyclo[3.1.0]hexane-2-d₁-2-carboxamide], 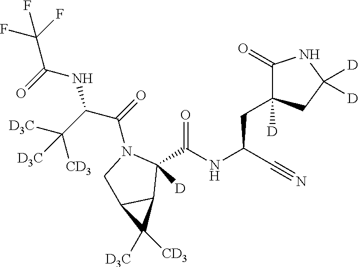 [SMILES: [2H]C1([2H])C[C@@]([2H])(C[C@@H](C#N)NC(=O)[C@]2([2H])[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,5,5-d₃)ethyl)-6,6-bis(methyl-d₃)-3-azabicyclo[3.1.0]hexane-2-d-2-carboxamide], 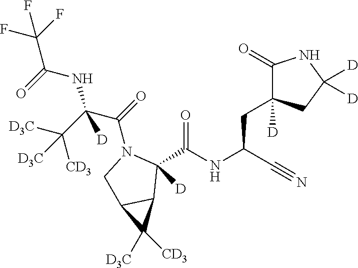 [SMILES: [2H]C1([2H])C[C@@]([2H])(C[C@@H](C#N)NC(=O)[C@]2([2H])[C@@H]3[C@H](CN2C(=O)[C@]([2H])(NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl-1-d)-6,6-bis(methyl-d₃)-3-azabicyclo[3.1.0]hexane-2-carboxamide], 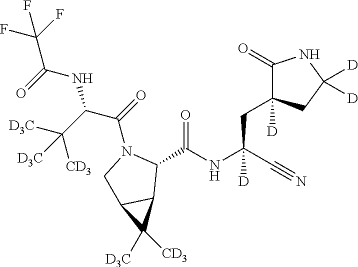
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-3,5,5-d₃)ethyl-1-d)-6,6-bis(methyl-d₃)-3-azabicyclo[3.1.0]hexane-2-d-2-carboxamide], 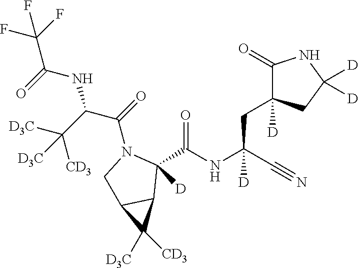
(1R,2S,5S)-3-((S)-3,3-bis(methyl-d₃)-2-(2,2,2-trifluoroacetamido)butanoyl-2,4,4,4-d₄)-N-((S)-1-cyano-2-((S)-2-oxopyrrolidin-3-yl-3,5,5-d₃)ethyl-1-d)-6,6-bis(methyl-d₃)-3-azabicyclo[3.1.0]hexane-2-d₁-2-carboxamide], 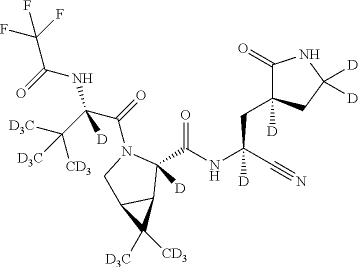
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4,5,5-d₄)ethyl-2,2-d₂)-6,6-bis(methyl-d₃)-3-azabicyclo[3.1.0]hexane-2-carboxamide], 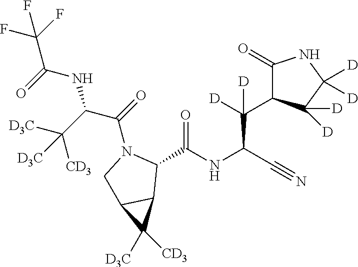 [SMILES: [2H]C([2H])([C@H]1C(=O)NC([2H])([2H])C1([2H])[2H])[C@@H](C#N)NC(=O)[C@@H]1[C@@H]2[C@H](CN1C(=O)[C@@H](NC(=O)C(F)(F)F)C(C([2H])([2H])[2H])(C([2H])([2H])[2H])C([2H])([2H])[2H])C2(C([2H])([2H])[2H])C([2H])([2H])[2H]]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-5,5-d₂)ethyl-2,2-d₂)-6,6-bis(methyl-d₃)-3-azabicyclo[3.1.0]hexane-2-carboxamide], 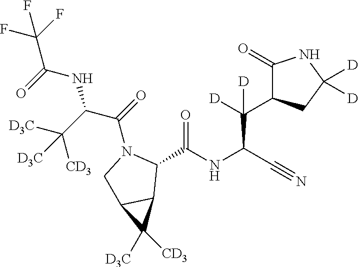 [SMILES: [2H]C1([2H])C[C@@H](C([2H])([2H])[C@@H](C#N)NC(=O)[C@@H]2[C@@H]3[C@H](CN2C(=O)[C@@H](NC(=O)C(F)(F)F)C(C([2H])([2H])[2H])(C([2H])([2H])[2H])C([2H])([2H])[2H])C3(C([2H])([2H])[2H])C([2H])([2H])[2H])C(=O)N1]
(1R,2S,5S)-3-((S)-3,3-bis(methyl-d₃)-2-(2,2,2-trifluoroacetamido)butanoyl-4,4,4-d₃)-N-((S)-1-cyano-2-((S)-2-oxopyrrolidin-3-yl-4,4-d₂)ethyl-2,2-d₂)-6,6-bis(methyl-d₃)-3-azabicyclo[3.1.0]hexane-2-carboxamide],

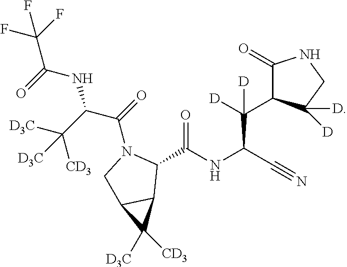

5. The method of claim 3 wherein a pharmaceutically effective amount of a compound of claim 3 is administered with a pharmaceutically effective amount of a compound selected from remdesivir, molnupiravir and monoclonal antibody cocktails.

6. The method of claim 5 wherein the monoclonal antibody cocktail is selected from bamlanivimab, bebtelovimab, casirivimab, cilgavimab, etesevimab, imdevimab, sotrovimab, and tixagevimab.

7. The method of claim 4 wherein a pharmaceutically effective amount of a compound of claim 4 is administered with a pharmaceutically effective amount of a compound selected from remdesivir, molnupiravir and monoclonal antibody cocktails.

8. The method of claim 7 wherein the monoclonal antibody cocktail is selected from bamlanivimab, bebtelovimab, casirivimab, cilgavimab, etesevimab, imdevimab, sotrovimab, and tixagevimab.

9. The method of claim 3 wherein a pharmaceutically effective amount of a compound of claim 3 is administered with a pharmaceutically effective amount of an angiotensin II receptor antagonist.

10. The method of claim 9 wherein the angiotensin II receptor antagonist is selected from Losartan, Valsartan, Candesartan, Irbesartan, Olemsartan, Telmisartan and Eprosartan.

11. The method of claim 4 wherein a pharmaceutically effective amount of a compound of claim 4 is administered with a pharmaceutically effective amount of an angiotensin II receptor antagonist.

12. The method of claim 11 wherein the angiotensin II receptor antagonist is selected from Losartan, Valsartan, Candesartan, Irbesartan, Olemsartan, Telmisartan and Eprosartan.

13. The method of claim 3 wherein a pharmaceutically effective amount of a compound of claim 3 is administered with a pharmaceutically effective amount of an angiotensin converting enzyme (ACE) inhibitor.

14. The method of claim 13 wherein the angiotensin converting enzyme inhibitor is selected from Enalapril, Lisinopril, Captopril, Benazepril, Fosinopril, Ramipril, Quinapril, and Perindopril.

15. The method of claim 3 wherein a pharmaceutically effective amount of a compound of claim 3 is administered with a pharmaceutically effective amount of a calcium channel antagonist.

16. The method of claim 15 wherein the calcium channel antagonist is selected from amlodipine, felodipine, Isradipine, Nicardipine, Nifedipine, Nimodipine and Nitrendipine.

17. The method of claim 3 wherein a pharmaceutically effective amount of a compound of claim 3 is administered with a pharmaceutically effective amount of an angiotensin converting enzyme-2 (ACE-2) inhibitor.

18. The method of claim 17 wherein the angiotensin converting enzyme-2 (ACE-2) inhibitor is selected from,
((S)-1-carboxy-2-(1-(3,5-dichlorobenzyl)-1H-imidazol-5-yl)ethyl)leucine-d₉ 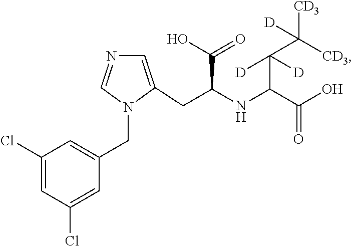 [SMILES: [2H]C([2H])([2H])C([2H])(C([2H])([2H])[2H])C([2H])([2H])C(N[C@@H](Cc1cncn1Cc1cc(Cl)cc(Cl)c1)C(=O)O)C(=O)O]
((S)-1-carboxy-2-(1-(3,5-dichlorobenzyl)-1H-imidazol-5-yl)ethyl)-L-leucine-d₉ 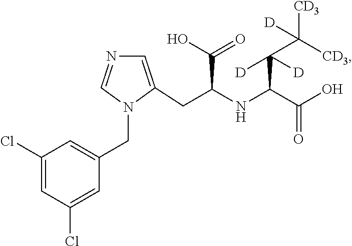 [SMILES: [2H]C([2H])([2H])C([2H])(C([2H])([2H])[2H])C([2H])([2H])[C@H](N[C@@H](Cc1cncn1Cc1cc(Cl)cc(Cl)c1)C(=O)O)C(=O)O]
((S)-1-carboxy-2-(1-(3,5-dichlorobenzyl)-1H-imidazol-5-yl)ethyl)-D-leucine-d₉ 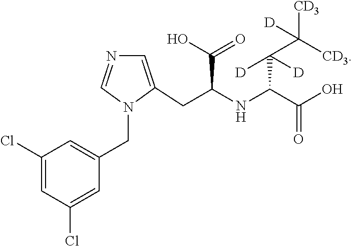 [SMILES: [2H]C([2H])([2H])C([2H])(C([2H])([2H])[2H])C([2H])([2H])[C@@H](N[C@@H](Cc1cncn1Cc1cc(Cl)cc(Cl)c1)C(=O)O)C(=O)O]
